(19) United States
(12) Patent Application Publication
(10) Pub. No.: US 2022/0218784 A1
(43) Pub. Date: Jul. 14, 2022

(54) Daptomycin Aqueous Formulations

(71) Applicant: XELLIA PHARMACEUTICALS APS, København S (DK)

(72) Inventors: Tina Gjoni, Zagreb (HR); Dubravka Strazic-Novakovic, Zagreb (HR); Ivona Jasprica, Zagreb (HR); Stipica Tomic, Zagreb (HR)

(21) Appl. No.: 17/610,108

(22) PCT Filed: May 8, 2020
(86) PCT No.: PCT/EP2020/062946

§ 371 Date: Nov. 9, 2021

Related U.S. Application Data
(60) Provisional application No. 62/846,038, filed on May 10, 2019.

(51) Int. Cl. A61K 38/12; A61K 47/02; A61K 47/10; A61K 9/08; A61K 47/22; A61K 47/20; A61K 47/18; A61K 47/26

(52) U.S. Cl. CPC A61K 38/12; A61K 47/02; A61K 47/10; A61K 9/08; A61K 47/183; A61K 47/20; A61K 47/186; A61K 47/26; A61K 47/22

(57) ABSTRACT

The present disclosure is related to providing a stable daptomycin aqueous pharmaceutical formulations as a contrast to the inconvenient and potentially problematic methods of lyophilized drug preparation and administration, wherein the aqueous formulations of daptomycin offer the advantage of ease of handling with a high degree of patient acceptability and compliance.

### CROSS-REFERENCE TO RELATED APPLICATIONS

This application claims the benefit of priority of U.S. Provisional Application No. 62/846,038, filed May 10, 2019, which is incorporated by reference herein in its entirety for any purpose.

### FIELD OF THE DISCLOSURE

The disclosure relates to aqueous pharmaceutical formulations comprising daptomycin.

### BACKGROUND

Lipopeptides represent a class of powerful anti-infective drugs, which exhibit highly effective antibacterial action against multidrug-resistant bacteria, as well as antifungal activity. Wide varieties of lipopeptide drugs, such as daptomycin, are now available on the market in order to fight invasive and often life-threatening infections.

Daptomycin is the first cyclic lipopeptide antibiotic approved by the U.S. Food and Drug Administration (FDA) in 2003 for the treatment of infections caused by Gram-positive pathogens, including methicillin- and vancomycin-resistant strains. Due to its unique mechanism of action, which is distinct from all other antimicrobial agents available in the market, daptomycin is able to overcome the mechanisms of resistance that many resistant strains have developed, and considering that rare incidences of clinical resistance to daptomycin are reported, the drug has become very important for current clinical practice.

Daptomycin (Structure 1) is composed of a decanoyl side chain attached to the N-terminus of a 13-amino acid peptide, wherein ten of the amino acids form a cyclic structure and three amino acids form a chain.

The cyclic section of the molecule is linked to the side chain through an ester bond between the C-terminal carboxyl group of kynurenine and the fourth residue (threonine).

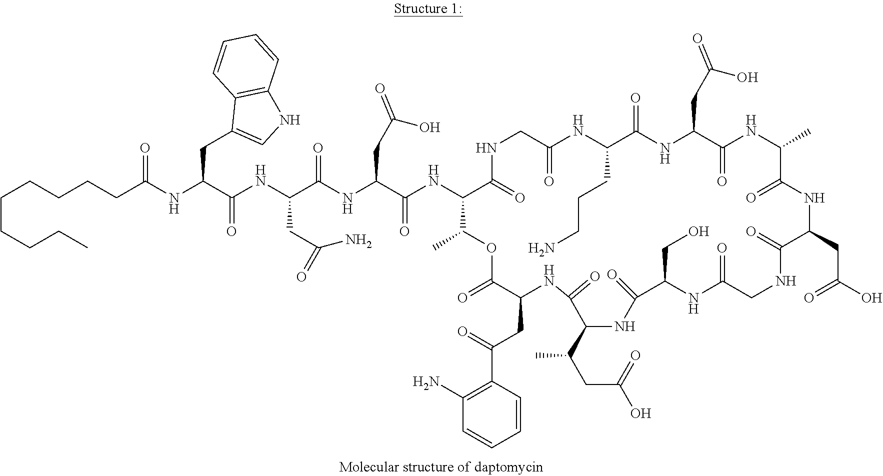 [SMILES: CCCCCCCCCC(=O)N[C@@H](Cc1c[nH]c2ccccc12)C(=O)N[C@@H](CC(N)=O)C(=O)N[C@@H](CC(=O)O)C(=O)N[C@@H]1C(=O)NCC(=O)N[C@@H](CCCN)C(=O)N[C@@H](CC(=O)O)C(=O)N[C@H](C)C(=O)N[C@@H](CC(=O)O)C(=O)NCC(=O)N[C@H](CO)C(=O)N[C@@H]([C@@H](C)CC(=O)O)C(=O)N[C@@H](CC(=O)c2ccccc2N)C(=O)O[C@@H]1C]

Daptomycin degrades upon exposure to liquid(s), especially water, to three main degradation products.

The first degradation product is identified as anhydrodaptomycin (Structure 2), which is formed by aspartyl transpeptidation at asp-9 residue.

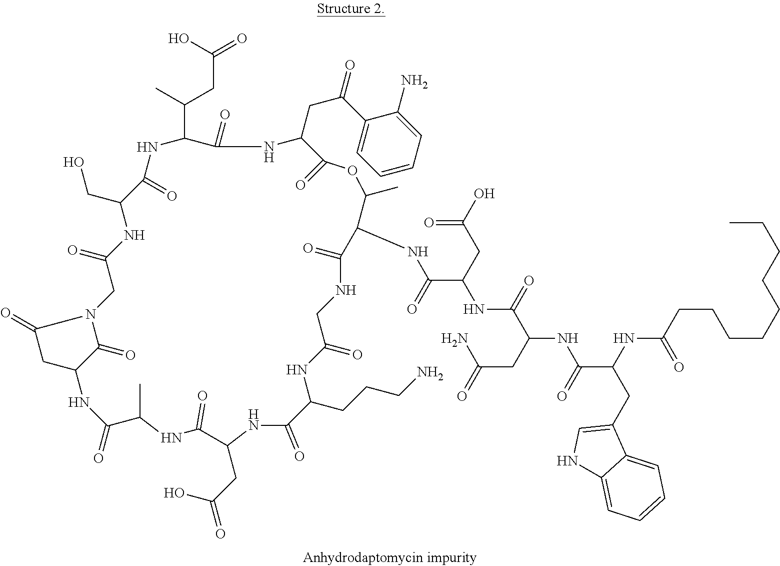 [SMILES: CCCCCCCCCC(=O)NC(Cc1c[nH]c2ccccc12)C(=O)NC(CC(N)=O)C(=O)NC(CC(=O)O)C(=O)NC1C(=O)NCC(=O)NC(CCCN)C(=O)NC(CC(=O)O)C(=O)NC(C)C(=O)NC2CC(=O)N(CC(=O)NC(CO)C(=O)NC(C(C)CC(=O)O)C(=O)NC(CC(=O)c3ccccc3N)C(=O)OC1C)C2=O]

The second undesirable product of daptomycin degradation shown in the Structure 3 is a beta (β-aspartyl) isomer formed with the rehydration of anhydrodaptomycin.

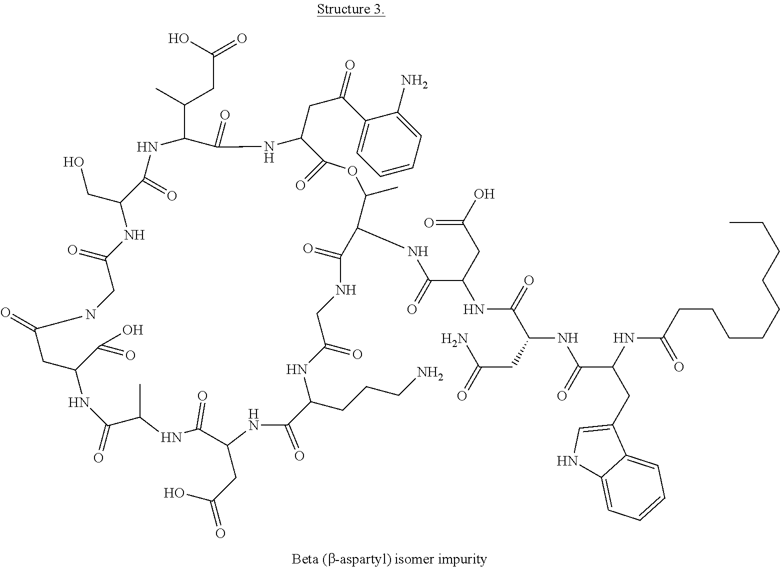 [SMILES: CCCCCCCCCC(=O)NC(Cc1c[nH]c2ccccc12)C(=O)N[C@H](CC(N)=O)C(=O)NC(CC(=O)O)C(=O)NC1C(=O)NCC(=O)NC(CCCN)C(=O)NC(CC(=O)O)C(=O)NC(C)C(=O)NC(C(=O)O)CC(=O)NCC(=O)NC(CO)C(=O)NC(C(C)CC(=O)O)C(=O)NC(CC(=O)c2ccccc2N)C(=O)OC1C]

According to Kirsch, L. E., Molloy, R. M., Debono, M. et al. Pharm Res (1989) 6 (5): 387-393 (hereinafter “Kirsch”), Muangsiri W, Kearney W R, Teesch L. Mm, Kirsch L. E., International Journal of Pharmaceutics. (2005) 289: 133-50 and Muangsiri W., Kirsch L. E. (2001) Journal of Pharmaceutical Sciences, 90 (8), pp. 1066-1075 (hereinafter “Muangsiri”), those two degradation pathways involve the formation of a succinimido intermediate (anhydrodaptomycin) formed by attack of carbonyl carbon of Asp9 side chain and subsequent reversible formation of two aspartic acid isomers formed by rehydration of the anhydrodaptomycin succinimide.

Another undesirable compound that daptomycin degrades to is lactone hydrolysis product (Structure 4).

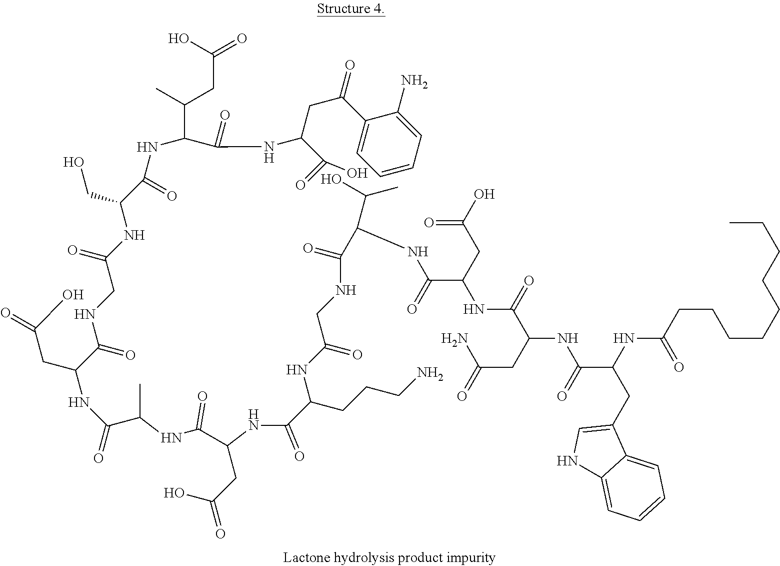 [SMILES: CCCCCCCCCC(=O)NC(Cc1c[nH]c2ccccc12)C(=O)NC(CC(N)=O)C(=O)NC(CC(=O)O)C(=O)NC(C(=O)NCC(=O)NC(CCCN)C(=O)NC(CC(=O)O)C(=O)NC(C)C(=O)NC(CC(=O)O)C(=O)NCC(=O)N[C@H](CO)C(=O)NC(C(=O)NC(CC(=O)c1ccccc1N)C(=O)O)C(C)CC(=O)O)C(C)O]

Kirsh and Muangsiri, additionally disclose that unknown, parallel pathways of daptomycin loss have been observed and are suggesting a transpeptidation degradation mechanism, which includes formation of succinimido intermediate, aspartyl ester hydrolysis and/or peptide bond cleavage/isomerization.

The degradation pathways of daptomycin under acidic, neutral, and alkaline conditions are known as ester hydrolysis occurring in alkaline conditions, aspartyl transpeptidation as the predominant pathway in the pH range of 3-6 and unknown degradation pathway that occurs at low pH. Besides the dependency of impurity formation on pH, impurity formation is also temperature dependent.

According to the available literature, it is extremely difficult to stabilize daptomycin in solutions, particularly aqueous solutions, due to the fact that daptomycin is susceptible to hydrolytic degradation and is known to degrade by aspartyl transpeptidation at the asp-9 residue in mildly acidic solutions.

In light of its instability in solution, daptomycin is currently commercially available only in the form of lyophilized powder for intravenous infusion (Cubicin® and Cubicin RF®) which requires reconstitution and subsequent dilution prior to patient administration.

Considering that daptomycin is administered intravenously on a daily basis during long-term treatment, and that the reconstitution step often takes of 30 minutes or even more, the lyophilized powder is not a convenient and practical form for medical professionals to handle.

Limited stability of reconstituted and diluted formulations is also a drawback of such a valuable drug. Currently, commercially available daptomycin has a maximum stability in the form of the reconstituted solution (i.e., daptomycin in an aqueous state) for 5 days in refrigerated conditions and 2 days in a room temperature.

Accordingly, formulations of daptomycin that do not require lyophilization and/or reconstitution and exhibit storage physico-chemical stability of typical solutions are needed. Furthermore, there is a need for aqueous formulations of daptomycin which exhibit typical storage physico-chemical stability and which are pharmaceutically acceptable, especially for parenteral administration.

Kirsch describe aqueous solutions of daptomycin with the pH range of 3 to 8, wherein daptomycin degradation product (anhydrodaptomycin and beta isomer) formations is investigated under different pH conditions.

Liquid compositions of daptomycin have been reported in WO2011062676 and WO2011035108, however those compositions comprise daptomycin in considerably lower concentrations of up to 25 mg/mL.

EP0386951 provides liquid formulations of daptomycin in different buffers, which allow daptomycin to be prepared in 5% dextrose and where in use the degradation of such prepared liquid formulations is approximately 1%-1.8% in only 24 h at 25° C. or in 7 days at 5° C., which is significantly reduced with respect to in use degradation of the known liquid formulations of daptomycin with 5% dextrose which is 15-20% degradation in the same period and under the same conditions.

WO2016059587 and WO2016059592 relate to stable, non-aqueous and ready-to-use injectable composition of daptomycin. However, according to the description, the water content of such formulations is less than 2%, since it is well known that daptomycin degrades at a fast rate in aqueous solutions.

WO2019043008 relates to lyophilized compositions of daptomycin which have good storage stability, but the document does not mention providing the liquid composition of the daptomycin that is stable in liquid form for a suitable time.

WO 2018073269 teaches that it is well known that daptomycin degrades at a fast rate in aqueous solutions.

Although some of the above documents are trying to solve daptomycin stability problems in the liquid/aqueous compositions, there is no stable aqueous composition of daptomycin in the market yet. Therefore, there is a need for stabile aqueous formulations of daptomycin.

### SUMMARY

Keeping in mind the current medical practitioners' needs and the problem of providing stable daptomycin liquid formulations, especially aqueous pharmaceutical formulations as a contrast to the inconvenient and potentially problematic methods of lyophilized drug preparation and administration, aqueous formulations of daptomycin were developed that offer the advantage of ease of handling with a high degree of patient acceptability and compliance.

It has been found that aqueous pharmaceutical formulations comprising daptomycin, calcium and at least one excipient possess surprisingly enhanced storage stability. Excipients include amino acids, sugars, sugar derivatives, saccharin, carboxylic acids and organic solvents, their pharmaceutically acceptable salts and derivatives thereof. It was found that, when daptomycin is formulated in solutions according to the present disclosure, degradation product formation is retarded, and accordingly, such solutions exhibit prolonged chemical and physical stability and provide more flexible storage conditions and handling when stored under refrigerated conditions, i.e. at a temperature of from 2° C. to 8° C. Furthermore, the aqueous pharmaceutical formulation of daptomycin in accordance with the present disclosure has improved stability under room temperature conditions, i.e. at a temperature 25° C.

This disclosure provides materials and methods related to aqueous pharmaceutical formulations of daptomycin. In some embodiments, an aqueous pharmaceutical formulation may comprise daptomycin, calcium, and at least one excipient. In some embodiments, the at least one excipient does not comprise a buffering agent. In some embodiments, the at least one excipient comprises at least one of PEG and/or glycerol.

In any of the above formulations, the aqueous pharmaceutical formulation may further comprise a pH range from pH 5.5 to pH 7.5.

In any of the above formulations, the calcium is provided in the form of calcium chloride (CaCl₂), Ca-α-D-heptagluconate, calcium saccharin, calcium lactate, or calcium acetate. In some of these embodiments, the calcium is in the form of calcium chloride. In other embodiments, the calcium is in the form of calcium saccharin. In some embodiments, the calcium is present in a molar ratio to daptomycin of 0.1:1 to 2:1. In some embodiments, the calcium is present in a molar ratio to daptomycin of 0.1:1 to 1:1.

In any of the above formulations, the at least one excipient may be selected from amino acid, sugar, sugar derivatives, saccharin, organic acids, organic solvents, betaine, taurine, nicotinamide or their pharmaceutically acceptable salts or derivatives thereof. In some embodiments, the at least one excipient is selected from organic solvents, such as alkyl alcohols, ethanol, benzyl alcohol, ethylene glycol, propylene glycol, butylene glycol, glycerol, polysorbates, for example polysorbate 20, polysorbate 40, and polysorbate 80, polyalkylene glycols, such as polyethylene glycol (PEG), polyethylene glycol 200 (PEG 200), polyethylene glycol 300 (PEG 300), polyethylene glycol 400 (PEG 400), polyethylene glycol 600 (PEG 600), polypropylene glycol, povidone and polybutylene glycol, and primary amides such as niacinamide. In other embodiments, the organic solvent is glycerol. In other embodiments, the organic solvent is polyethylene glycol 400 (PEG 400). In some embodiments, the formulation comprises two or more organic solvents. In such embodiments, the formulation comprises glycerol and PEG 400. In various embodiments wherein the formulation comprises organic solvent, each organic solvent in the formulation is in the amount of 20% V/V or less. In some embodiments wherein the organic solvent is glycerol, the glycerol is in the formulation in the amount of 10% V/V or less. In some embodiments wherein the organic solvent is PEG 400, the PEG 400 in the formulation in the amount of 10% V/V or less.

In any of the above formulations, the daptomycin is at a concentration of from 0.5 mg/mL to 500 mg/mL. In some embodiments, the daptomycin is at a concentration of from 2 mg/ml to 20 mg/ml. In some embodiments, the daptomycin is at a concentration of 50 mg/ml.

In an embodiment wherein an aqueous pharmaceutical formulation comprises 50 mg/ml of daptomycin, the pharmaceutical formulation may further comprise (1) daptomycin, calcium and PEG 400; (2) a molar ratio of daptomycin to calcium of 1:1; (3) PEG 400 at a concentration of 10% V/V or less; and (4) a formulation pH of 7.

In an embodiment wherein an aqueous pharmaceutical formulation comprises 50 mg/ml of daptomycin, the pharmaceutical formulation may further comprise (1) daptomycin, calcium and glycerol; (2) a molar ratio of daptomycin to calcium of 1:1; (3) glycerol at a concentration of 10% V/V or less; and (4) a formulation pH of 7.

In an embodiment wherein an aqueous pharmaceutical formulation comprises 50 mg/ml of daptomycin, the pharmaceutical formulation may further comprise (1) daptomycin, calcium, PEG 400 and glycerol; (2) a molar ratio of daptomycin to calcium of 1:1; (3) PEG 400 at a concentration of 10% V/V or less; (4) glycerol at a concentration of 10% V/V or less; and (5) a formulation pH of 7.

In any of the above formulations, the aqueous pharmaceutical formulation comprises at least 50% water V/V. In some embodiments, the aqueous pharmaceutical formulation comprises more than 50% water V/V. In some embodiments, the aqueous pharmaceutical formulation comprises at least 60% water V/V. In some embodiments, the aqueous pharmaceutical formulation comprises at least 50%, 60%, 70%, 80%, 85%, 90%, 95%, 98%, or 99% water V/V.

The disclosure also relates to the packaging any of the above aqueous pharmaceutical formulations. In some embodiments, the aqueous pharmaceutical formulation may be packaged in a vial for dilution prior to administration a patient. In some embodiments, the aqueous pharmaceutical formulation is stable for at least 4 days at temperatures of 30° C.

This disclosure also relates to the use of any of the above aqueous pharmaceutical formulations. In some embodiments, the aqueous pharmaceutical formulation may be used in the treatment of microbial infections caused by Gram positive bacteria. In some embodiments, the aqueous pharmaceutical formulation is used in the treatment of skin and soft-tissue infections (cSSTI) or Staphylococcus aureus bloodstream infections (bacteremia).

This disclosure also provides process for manufacturing any of the above aqueous pharmaceutical formulations. In various embodiments, the process may comprise the steps of mixing of daptomycin, calcium and at least one excipient into solution, adjusting the pH of such solution to pH from 5.5 to 7.5 with a suitable pH adjusting agent.

Any of the above aqueous pharmaceutical formulations may be used to treat a patient with a microbial infection by administering the aqueous pharmaceutical formulation, and optionally diluting the pharmaceutical formulation before administering it to the patient. In some embodiments, the aqueous pharmaceutical formulation is diluted before administering it to the patient.

It is to be understood that both the foregoing description and the following further description are exemplary and explanatory only and are not restrictive of the claims.

### DETAILED DESCRIPTION

Provided herein are aqueous pharmaceutical formulations comprising daptomycin, calcium and at least one excipient selected from amino acids, sugars, sugar derivatives, saccharin, carboxylic acids and organic solvents, their pharmaceutically acceptable salts and derivatives thereof. Compositions according to the present disclosure showed surprising stability for a reasonable period of time, when stored at a temperature of from 2° C. to 8° C., such as, e.g., at a temperature of 2° C., 3° C. or less, 4° C. or less, 5° C. or less, 6° C. or less, 7° C. or less or 8° C. or less.

Term “stable” as used herein, refers to a pharmaceutical formulation containing daptomycin having sufficient stability to have utility as a pharmaceutical product, i.e. that the aqueous pharmaceutical formulation exhibits an acceptable amount of daptomycin being present, or an acceptable amount of daptomycin having degraded after a certain period of time. Accordingly, in a stable solution or formulation unacceptable degradation of daptomycin is avoided, while pharmaceutically desirable appearance such as acceptable color, clarity and no visible particles (for example, no visible particles to the naked eye) is retained.

Regarding stability of a pharmaceutical solution or composition, as little degradation of the active ingredient as possible is an important factor.

Another important factor with respect to the pharmaceutical solution is the formation of impurities, which may be formed by degradation of the active ingredient.

Accordingly, “stability” may also be defined by the amount of total or specific individual impurities generated after a suitable period of time. By the specific individual impurities, it is predominantly meant three main daptomycin degradation products, i.e. anhydrodaptomycin, beta (β-aspartyl) isomer impurity and lactone hydrolysis product impurity (Structures 2-4).

The stability can also be presented as an increase of total or specific impurities in certain time point with respect to the initial specific impurity amount.

The amount of impurities being present may be expressed as a percentage, for example as a peak-area percentage of a HPLC chromatogram.

The disclosed formulations exhibit acceptable stability with regard to retaining the daptomycin efficacy and potency in solution dosage form, avoid unacceptable degradation of active substance to undesired related substances, and retain pharmaceutically desirable appearance such as acceptable color, clarity and no visible particles.

As used herein, “stable” is defined either as no more than 10% of increase of total impurities formation, determined by HPLC analysis, or as no more than 5% of increase of every individual impurity formation, determined by HPLC analysis, under typical storage conditions.

For example, a stable or stabilized solution can be one which has not more than 1%, 2%, 3%, 4%, 5%, 6%, 7%, 8%, 9%, 10%, of increase of total impurities formation after a predetermined time period.

Further, a stable or stabilized solution can be one that has not more than 1%, 2%, 3%, 4%, 5% increase of every individual impurity formation after a predetermined time period.

The aqueous solutions of daptomycin described herein are stable over time periods of 7 days (1 week), 14 days (2 weeks), 30 days (1 month), 60 days (2 months), 3 months, 4 months, 180 days (6 months), 9 months, 12 months (1 year) and longer at temperatures of 2-8° C.

The aqueous solutions of daptomycin described herein are stable for at least 4 days at temperature of 30° C.

In one aspect, a stable or stabilized solution can be one that has not more than 5% increase of every individual impurity formation over 12 months and longer at temperatures of 2-8° C.

In one aspect, a stable or stabilized solution can be one that has not more than 5% increase of anhydrodaptomycin impurity formation after 12 months and longer at temperatures of 2-8° C.

In one aspect, a stable or stabilized solution can be one that has not more than 2% increase of beta (β-aspartyl) isomer formation after 6 months and longer at temperatures of 2-8° C.

In one aspect, a stable or stabilized solution can be one that has not more than 4% increase of beta (β-aspartyl) isomer formation after 9 months and longer at temperatures of 2-8° C.

In one aspect, a stable or stabilized solution can be one that has not more than 5% increase of beta (β-aspartyl) isomer formation after 12 months and longer at temperatures of 2-8° C.

Analysis of the aqueous formulations described herein can be performed using techniques known in the art, including HPLC.

By terms “pharmaceutical composition” or “pharmaceutically acceptable composition” as used herein, is meant any composition suitable and intended for in vivo use, for example administration to a patient or a subject. As used herein, the terms “patient” and “subject” are interchangeable, and refer to any human or animal individual who is receiving a composition as described herein.

As used herein, the terms “pharmaceutical composition”, “pharmaceutical formulation”, “composition” and “formulation” are used interchangeably.

As used herein, “aqueous composition” or “aqueous solutions” means any solution in which water is the main solvent (equal or above 50% V/V). Aqueous solutions include, but are not limited to solutions comprising at least 50%, 60%, 70%, 80%, 85%, 90%, 95%, 98% or 99% V/V water. Aqueous solutions can comprise a pharmaceutically acceptable organic solvent like ethanol, glycerol, propylene glycol, polyethylene glycols (PEG 200, PEG 300, PEG 400, 20 PEG 600, PEG 4000, etc.). Aqueous solutions can comprise 50% V/V or less of a pharmaceutically acceptable organic solvent.

In an aspect, the calcium in the formulations is added in the form of calcium chloride (CaCl₂), Ca-α-D-heptagluconate, calcium lactate, calcium saccharin, calcium acetate, or a combination thereof. In an aspect, the calcium is added in the form of calcium saccharin. Surprisingly, it has been found that calcium saccharin provides a good stability effect to aqueous pharmaceutical formulations of daptomycin.

In an aspect, daptomycin is present in a molar ratio to calcium of 1:0.1 to 1:2.

In an aspect, daptomycin is present in the molar ratio to calcium of 1:0.1 to 1:1.

In specific aspects, daptomycin is present in a molar ratio to calcium of 1:0.1, 1:0.5, 1:1, 1:1.5 and 1:2.

In an aspect, the pH of formulations is from 5.5 to 7.5. In specific aspects, the pH of formulations is from 6.0 to 7.2. In an aspect, the pH of formulations is 7.0.

The pH in can be adjusted using pH adjusters known in the state of art. The term “pH adjuster” means a compound or composition used to change the pH of a formulation to a targeted pH value or pH range. For example, pH adjusters include hydrochloric acid, sulphuric acid, sodium hydroxide and ammonium hydroxide.

The term “pH buffer” means a compound or composition used to maintain the pH value or pH range of a formulation within desired parameters over time. pH adjusters do not maintain the pH value or pH range within desired parameters over time. pH buffers include aqueous solutions comprising a mixture of a weak acid and its conjugate base (or a weak base and its conjugate acid). The pH of a buffered solution changes very little when a small amount of an acid or base is added to it. Examples of pH buffers include carbonate buffer, citrate buffer, phosphate buffer and so called “biological buffers” such as for example ADA, ACES, MES, TRIS, PIPES, MOPS, HEPES.

It is discovered that formulations do not require additional pH control and hence no buffers are needed. Thus, in some embodiments, the formulation does not include pH buffers including carbonate buffer, citrate buffer, phosphate buffer or so called “biological buffers” such as for example ADA, ACES, MES, TRIS, PIPES, MOPS, or HEPES.

In an aspect, further excipients include organic acids (other than already mentioned carboxylic acids), trimethylglycine (hereinafter “betaine”), taurine, nicotinamide, spermine, spermidine, their pharmaceutically acceptable salts and derivatives thereof.

As used herein, “pharmaceutically acceptable” is meant that they are useful in preparing a pharmaceutical composition that is generally non-toxic and neither biologically nor otherwise undesirable, further that they do not cause unacceptable loss of pharmacological activity of the drug in question, and are acceptable for use in treatment of humans and/or animals.

In an aspect, aqueous pharmaceutical formulations comprise daptomycin, calcium and at least one amino acid are provided herein.

According to this aspect, the amino acid comprises non-naturally and naturally occurring amino acids, both of L- and D-orientation, proteinogenic and non-proteinogenic, including any salts thereof and chemically modified amino acids by e.g. acetylation or formylation.

In one aspect, the amino acids are L-oriented.

Exemplary amino acids include alanine, asparagine, aspartic acid, glutamine, glutamic acid, glycine, leucine, methionine, ornithine, phenylalanine, proline, serine, tryptophan, tyrosine, valine and their pharmaceutically acceptable salts or derivatives thereof, and combinations thereof. Derivatives of amino acids include N-formyl-glycine, N-acetyl-D-alanine, N-acetyl-L-alanine, and pharmaceutically acceptable salts thereof, and combinations thereof.

In one aspect, amino acids include alanine, glutamic acid, glycine, leucine, phenylalanine, proline, tryptophan, tyrosine, and combinations thereof.

In one aspect, the at least one amino acid comprises polar and/or aliphatic amino acids, such as serine and leucine.

In another aspect, the at least one amino acid comprises aromatic and/or cyclic amino acids, such as tyrosine, phenylalanine, tryptophan and proline.

In an aspect, the amino acid comprises proline, tyrosine, tryptophan, leucine, serine, phenylalanine, or a combination thereof.

In an aspect, the amino acid comprises L-proline, L-tyrosine, L-tryptophan, L-leucine, L-serine, L-phenylalanine, or a combination thereof.

In one aspect, the at least one amino acid is L-tyrosine.

In one aspect, the at least one amino acid is L-proline.

According to one aspect, the formulation may comprise two or more amino acids or their pharmaceutically acceptable salts or derivatives thereof.

The second or any further amino acid comprises alanine, asparagine, aspartic acid, glutamine, glutamic acid, glycine, leucine, methionine, ornithine, phenylalanine, proline, serine, tryptophan, tyrosine, valine or its pharmaceutically acceptable salts or derivatives thereof. Amino acid derivatives include N-formyl-Glycine, N-acetyl-D-alanine, N-acetyl-L-alanine and pharmaceutically acceptable salts thereof.

In one aspect, the second or any further amino acid is selected from proline, tyrosine, tryptophan, leucine, serine and phenylalanine.

In another aspect, the second amino acid is selected from L-proline, L-tyrosine, L-leucine, L-tryptophan, L-serine and L-phenylalanine.

In one aspect, the formulation comprises daptomycin, calcium and two amino acids.

In one aspect, the second amino acid is L-proline or L-tyrosine.

In one aspect, the second amino acid is L-tyrosine.

In an aspect, the formulation comprises daptomycin, calcium and two amino acids, wherein the first amino acid is L-proline and the second amino acid is L-tyro sine.

The molar ratio of daptomycin to each of the amino acids is from 1:0.01 to 1:10.

In an aspect, the molar ratio of daptomycin to each of the amino acids is from 1:0.02 to 1:1.

The inventors have surprising found that, in combination with calcium, an amino acid, specifically in in low amounts, provides a better stabilization effect to aqueous formulations of daptomycin having a higher amount of the same amino acid. This is in contrast to the teaching of prior art relating to stabilization of lyophilized formulations of daptomycin, wherein the higher molar ratio of amino acids provided better results with respect to stability of the formulation.

By “low amount” of amino acid it is meant an amount of each amino acid, which is in molar ratio to daptomycin lower than of 0.5:1.

Therefore, in an aspect, the molar ratio of daptomycin to each of the amino acids is from 1:0.02 to 1:0.4. Thus, formulations according to the present disclosure include molar ratios of daptomycin to each of the amino acids of 1:0.02, 1.0.03, 1:0.04, 1:0.05, 1:0.06, 1:0.07, 1:0.08, 1:0.09, 1:0.1, 1:0.2, 1:0.3, 1:0.4.

According to an aspect, the formulation comprises daptomycin, calcium and at least one organic solvent, such as alkyl alcohols, ethanol, benzyl alcohol, ethylene glycol, polyvinylalcohol, propylene glycol, butylene glycol, glycerin, glycerol, polysorbates, such as polysorbate 20, polysorbate 40, polysorbate 80, polyalkylene glycols, such as polyethylene glycol (PEG), polyethylene glycol 200 (PEG 200), polyethylene glycol 300 (PEG 300), polyethylene glycol 400 (PEG 400), polyethylene glycol 600 (PEG 600), polypropylene glycol, povidone, polybutylene glycol, primary amides such as niacinamide, and combinations thereof.

In an aspect, the at least one organic solvent includes povidone, glycerol, polyethylene glycols, and combinations thereof. In an aspect, the pharmaceutical composition comprises at least one organic solvent and is having pH from 6.0 to 7.2. In an aspect, the pharmaceutical composition comprises at least one organic solvent, is having pH from 6.0 to 7.2 and is having at least 80% V/V of water.

In an aspect, the polyethylene glycol is PEG 400.

In an aspect, at least one organic solvent is glycerol.

In an aspect, the pharmaceutical composition comprises two or more organic solvents. In an aspect, the pharmaceutical composition comprises daptomycin, calcium and two organic solvents, which are glycerol and PEG 400.

In an aspect, the pharmaceutical formulation comprises glycerol and PEG 400 and is having pH from 6.0 to 7.2. In an aspect, the pharmaceutical composition comprises glycerol and PEG 400 and is having pH 7.0.

In an aspect, the formulations will comprise less than 50% V/V of organic solvents in total. Specifically, in an aspect, the formulations will comprise less than 20% V/V, of each organic solvent.

In an aspect, the pharmaceutical formulation comprises glycerol and PEG 400 and glycerol and PEG 400 are each comprised in the formulation in concentration of 20% V/V or less and wherein the formulation has a pH from 6.0 to 7.2.

In an aspect, the pharmaceutical formulation comprises glycerol and PEG 400 and glycerol and PEG 400 are each comprised in the formulation in concentration of 20% V/V or less and wherein the formulation has a pH of 7.

In an aspect, the pharmaceutical formulation comprises glycerol and PEG 400 and glycerol and PEG 400 are in total comprised in the formulation in concentration of 20% V/V or less and wherein the formulation has a pH of 7.

In an aspect, the aqueous formulation comprises 13% V/V or less of PEG 400. In an aspect, the aqueous formulation comprises 10% V/V or less of PEG 400. In an aspect, the PEG 400 is comprised in the formulation in an amount of 9.5% V/V or less. In further aspect, PEG 400 is comprised in the formulation of 8% V/V, 7% V/V, 6% V/V, 5% V/V, 4% V/V, 3% V/V, 2% V/V, 1.5% V/V or 1% V/V or less.

In an aspect, the pharmaceutical composition comprises 5% V/V or less of PEG 400, is having pH from 6.0 to 7.2 and is having at least 80% V/V of water.

In an aspect, the pharmaceutical composition comprises 5% V/V or less of PEG 400, is having pH 7 and is having at least 90% V/V of water.

In an aspect, glycerol is comprised in the formulation of 20% V/V, 19% V/V, 18% V/V, 17% V/V, 16% V/V, 15% V/V, 14% V/V, 13% VAT, 12% VAT, 10% VAT, 11% VAT, 10% V/V, 9% VAT, 8% V/V, 7% V/V, 6% VAT, 5% VAT, 4% VAT, 3% VAT, 2% VAT, 1.5% V/V or 1% V/V or less.

In an aspect, the aqueous formulation comprises 10% VAT or less of glycerol. In an aspect, the glycerol is comprised in the formulation in the amount of 8% VAT or less. In an aspect, the glycerol is comprised in the formulation in the amount of 5% VAT or less.

In an aspect, the pharmaceutical composition comprises 5% V/V or less of glycerol, is having pH from 6.0 to 7.2 and is having at least 80% VAT of water.

In an aspect, the pharmaceutical composition comprises 5% V/V or less of glycerol, is having pH 7 and is having at least 90% VAT of water.

In an aspect, the pharmaceutical formulation comprises daptomycin, calcium, glycerol and PEG 400, where concentration of glycerol in 16% VAT or less and the concentration of PEG 400 is 15% V/V or less.

In an aspect, the pharmaceutical formulation comprises daptomycin, calcium, glycerol and PEG 400, where concentration of glycerol in 5% V/V or less and the concentration of PEG 400 is 5% V/V or less.

In an aspect, the pharmaceutical formulation comprises daptomycin, calcium, glycerol and PEG 400, where concentration of glycerol in 13% VAT or less and the concentration of PEG 400 is 5% V/V or less.

In an aspect, the pharmaceutical formulation comprises daptomycin, calcium, glycerol and PEG 400, where concentration of glycerol in 5% VAT or less and the concentration of PEG 400 is 13% V/V or less.

In an aspect, the pharmaceutical formulation comprises daptomycin, calcium, glycerol, PEG 400 and at least 80% VAT of water. In an aspect, the pharmaceutical formulation comprises daptomycin, calcium, glycerol, PEG 400 and at least 90% VAT of water.

In an aspect, the pharmaceutical formulation comprises daptomycin, calcium, PEG 400 and at least 90% V/V of water.

In an aspect, the pharmaceutical formulation comprises daptomycin, calcium and glycerol, wherein formulation comprises 50 mg/ml of daptomycin; the molar ratio of daptomycin to calcium is 1:1, glycerol is comprised in concentration of 10% V/V or less and wherein the pH of the formulation is 7.0.

In an aspect, the pharmaceutical formulation comprises daptomycin, calcium and PEG, wherein formulation comprises 50 mg/ml of daptomycin; the molar ratio of daptomycin to calcium is 1:1, PEG is comprised in concentration of 10% V/V or less and wherein the pH of the formulation is 7.0. In some embodiments, the PEG is PEG 400.

In an aspect, the pharmaceutical formulation comprises daptomycin, calcium and PEG, wherein formulation comprises 50 mg/ml of daptomycin; the molar ratio of daptomycin to calcium is 1:1, PEG is comprised in concentration of 5% V/V or less and wherein the pH of the formulation is 7.0. In some embodiments, the PEG is PEG 400.

In an aspect, the pharmaceutical formulation comprises daptomycin, calcium, glycerol and PEG, wherein formulation comprises 50 mg/ml of daptomycin; the molar ratio of daptomycin to calcium is 1:1, PEG is comprised in concentration of 10% V/V or less, glycerol is comprised in concentration of 10% V/V or less and wherein the pH of the formulation is 7.0. In some embodiments, the PEG is PEG 400.

In an aspect, the pharmaceutical formulation comprises daptomycin, calcium, glycerol and PEG, wherein formulation comprises 50 mg/ml of daptomycin; the molar ratio of daptomycin to calcium is 1:1, PEG is comprised in concentration of 5% V/V or less, glycerol is comprised in concentration of 5% V/V or less and wherein the pH of the formulation is 7.0. In some embodiments, the PEG is PEG 400.

In some embodiments, the aqueous solutions include, but are not limited to solutions comprising at least 50%, 60%, 70%, 80%, 85%, 90%, 95%, 98% or 99% V/V water. In some embodiments, the formulation comprises 60% V/V or more water.

In an aspect, the aqueous formulation comprises daptomycin, calcium, at least one amino acid and at least one organic solvent.

In an aspect, the aqueous formulation comprises daptomycin, calcium, one or two amino acids and one or two organic solvents.

According to the present disclosure, the aqueous pharmaceutical formulations may comprise saccharin and/or one or more pharmaceutically acceptable salts or derivatives thereof.

As used herein, by the term “saccharin”, it is meant saccharin, its pharmaceutically acceptable salts and derivatives thereof.

In an aspect, pharmaceutically acceptable salts of saccharin include positively charged ions, such as mono or divalent cations. In an aspect, positively charged ions of pharmaceutically acceptable salts of saccharin include Ca, Na, Mg, or K cations.

In an aspect, the molar ratio of daptomycin to saccharin, is from 1:0.1 to 1:3. In an aspect, the molar ratio of daptomycin to saccharin is from 1:0.2 to 1:1. In an aspect, the molar ratio of daptomycin to saccharin is 1:0.5.

In an aspect, the aqueous formulation comprises at least one carboxylic acid, its pharmaceutically acceptable salts or derivatives thereof. Carboxylic acids include lactic, citric, succinic and gluconic acids. Salts of carboxylic acids include calcium, magnesium, and sodium salts.

In an aspect, the salts of carboxylic acids are selected from Na-L-lactate and Na-gluconate.

In an aspect, the molar ratio of daptomycin to each of carboxylic acids is from 1:0.05 to 1:1.

In an aspect, the aqueous formulation comprises daptomycin, calcium, at least one amino acid, at least one organic solvent and at least one carboxylic acid, its salt or derivative thereof.

In an aspect, the aqueous formulation comprises daptomycin, calcium, one or two amino acids and one or two organic solvents and one or two carboxylic acids, their salts or derivatives thereof.

In other aspect, the aqueous formulation comprises a sugar derivative. In some embodiments, sugar derivatives are halogenated sugar derivatives. In some embodiments, the halogenated sugar derivative is sucralose.

In an aspect, the molar ratio of daptomycin to sugar derivative is from 1:0.05 to 1:10.

In an aspect, the molar ratio of daptomycin to sucralose is from 1:0.05 to 1:5. In another aspect, the molar ratio of daptomycin to sucralose is from 1:0.05 to 1:10.

Furthermore, in other aspect, the aqueous formulation further comprises a sugar. In an aspect, the sugar is non-reducing sugar such as sucrose, trehalose, raffinose, dextran, and combinations thereof.

In an aspect, the at least one sugar is sucrose or trehalose.

In an aspect, the at least one sugar is sucrose.

In an aspect, the at least one sugar is trehalose.

In an aspect, the aqueous formulation comprises two sugars, where the first sugar is sucrose and the second sugar is trehalose.

In an aspect, the molar ratio of daptomycin to each of selected sugars is from 1:0.5 to 1:20.

In an aspect, the molar ratio of daptomycin to each of selected sugars is from 1:1 to 1:10.

In an aspect, the molar ratio of daptomycin to sucrose or trehalose is from 1:4 to 1:10.

In an aspect, the molar ratio of daptomycin to raffinose is from 1:1 to 1:10.

In an aspect, the aqueous formulation comprises daptomycin, calcium, at least one amino acid, at least one organic solvent, and at least one sugar.

In an aspect, the aqueous formulation comprises daptomycin, calcium, one or two amino acids, one or two organic solvents, and one or two sugars.

In an aspect, the aqueous formulation comprises daptomycin, calcium, at least one amino acid, at least one organic solvent, at least one sugar, and at least one carboxylic acid, its salt or derivative thereof.

In an aspect, the aqueous formulation comprises daptomycin, calcium, one or two amino acids and one or two organic solvents, one or two sugars and one or two carboxylic acids, their salts or derivatives thereof.

On an aspect, the molar ratio of daptomycin to an excipient selected from organic acids, betaine, spermine, spermidine, taurine and nicotinamide, is from 1:0.05 to 1:20.

In an aspect, the aqueous pharmaceutical formulation comprises daptomycin, calcium, such as calcium saccharine in a molar ratio of calcium to daptomycin of 1:1, an amino acid L-proline in a molar ratio to daptomycin of 0.1:1, an amino acid L-tyrosine in a molar ratio to daptomycin of 0.05:1, glycerol in an amount of 8% V/V, PEG 400 in an amount of 10% V/V and sucrose in a molar ratio to daptomycin of 9.5:1, and wherein the composition has a pH of 6.3.

In an aspect, the aqueous pharmaceutical formulation comprises daptomycin, calcium saccharine in a molar ratio of calcium to daptomycin of 1:1, an amino acid L-proline in a molar ratio to daptomycin of 0.05:1, an amino acid tyrosine in a molar ratio to daptomycin of 0.05:1, glycerol in an amount of 8% V/V, PEG 400 in an amount of 10% V/V and sucrose in molar ratio to daptomycin of 5:1 and wherein the composition has a pH of 7.2.

In an aspect, the aqueous pharmaceutical composition comprises daptomycin, calcium chloride in a molar ratio of calcium to daptomycin of 1:1, an amino acid L-proline in a molar ratio to daptomycin of 0.05:1, an amino acid L-tyrosine in a molar ratio to daptomycin of 0.05:1, glycerol in an amount of 8% V/V, PEG 400 in an amount of 10% V/V and sucrose in a molar ratio to daptomycin of 5:1 and wherein the composition has a pH of 7.2.

In an aspect, the aqueous pharmaceutical composition comprises daptomycin, calcium saccharin in a molar ratio of calcium to daptomycin of 1:1, an amino acid L-tyrosine in a molar ratio to daptomycin of 0.05:1, glycerol in an amount of 8% V/V, PEG 400 in an amount of 10% V/V and sucrose in a molar ratio to daptomycin of 5:1, and wherein the composition has a pH of 7.2.

In an aspect, the aqueous pharmaceutical composition comprises daptomycin, calcium chloride in a molar ratio to of calcium daptomycin of 1:1, an amino acid L-tyrosine in a molar ratio to daptomycin of 0.05:1, glycerol in an amount of 8% V/V, PEG 400 in an amount of 10% V/V and sucrose in a molar ratio to daptomycin of 5:1, and wherein the composition has a pH of 7.

In an aspect, the aqueous pharmaceutical composition comprises daptomycin, calcium, such as calcium saccharine in a molar ratio of calcium to daptomycin of 1:1, an amino acid L-tyrosine in a molar ratio to daptomycin of 0.05:1, Na-L-lactate in a molar ratio to daptomycin of 0.05:1, glycerol in an amount of 8% V/V, PEG 400 in an amount of 10% V/V and sucrose in a molar ratio to daptomycin of 5:1, and wherein the composition has a pH of 7.

In an aspect, the aqueous pharmaceutical composition comprises daptomycin, calcium saccharine in a molar ratio of calcium to daptomycin of 1:1, an amino acid L-tyrosine in a molar ratio to daptomycin of 0.05:1, Na-L-lactate in a molar ratio to daptomycin of 0.05:1, glycerol in an amount of 8% V/V, PEG 400 in an amount of 10% V/V, and wherein the composition has a pH of 7.

In an aspect, the aqueous pharmaceutical composition comprises daptomycin, calcium, such as calcium saccharin in a molar ratio to daptomycin of 1:1, an amino acid L-tyrosine in a molar ratio to daptomycin of 0.05:1, Na-L-lactate in a molar ratio to daptomycin of 0.05:1, glycerol in an amount of 8%, and sucrose in a molar ratio to daptomycin of 5:1, and wherein the composition has a pH of 7.

In an aspect, the aqueous pharmaceutical composition comprises daptomycin, calcium saccharin in a molar ratio to daptomycin of 1:1, an amino acid L-tyrosine in a molar ratio to daptomycin of 0.05:1, Na-gluconate in a molar ratio to daptomycin of 0.05:1, glycerol in an amount of 8%, and wherein the composition has a pH of 7.2.

In an aspect, the aqueous pharmaceutical composition comprises daptomycin, calcium chloride in a molar ratio of calcium to daptomycin of 1:1, an amino acid L-tyrosine in a molar ratio to daptomycin of 0.05:1, Na-L-lactate in a molar ratio to daptomycin of 0.1:1, glycerol in an amount of 8% V/V, PEG 400 in an amount of 10% V/V, and sucrose in a molar ratio to daptomycin of 5:1, and wherein the composition has a pH of 7.

In an aspect, the aqueous pharmaceutical composition comprises daptomycin, calcium saccharine in a molar ratio of calcium to daptomycin of 1:1, an amino acid L-tyrosine in a molar ratio to daptomycin of 0.05:1, Na-L-lactate in a molar ratio to daptomycin of 0.1:1, glycerol in an amount of 8% V/V, PEG 400 in an amount of 10% V/V, and sucrose in a molar ratio to daptomycin of 5:1, and wherein the composition has a pH of 7.2.

In an aspect, the aqueous pharmaceutical composition comprises daptomycin, calcium chloride in a molar ratio of calcium to daptomycin of 1:1, an amino acid L-tyrosine in a molar ratio to daptomycin of 0.05:1, glycerol in an amount of 8% VN, PEG 400 in an amount of 10% V/V, and D(+)trehalose in a molar ratio to daptomycin of 5:1, and wherein the composition has a pH of 7.

In an aspect, the aqueous pharmaceutical composition comprises daptomycin, calcium saccharin in a molar ratio of calcium to daptomycin of 1:1, an amino acid L-tyrosine in a molar ratio to daptomycin of 0.05:1, glycerol in an amount of 8% V/V, PEG 400 in an amount of 10% V/V, D(+)trehalose in a molar ratio to daptomycin of 5:1, and Na-L-lactate in a molar ratio to daptomycin of 0.05:1, and wherein the composition has a pH of 7.

In an aspect, the aqueous pharmaceutical composition comprises daptomycin, calcium chloride in a molar ratio of calcium to daptomycin of 1:1, an amino acid L-proline in a molar ratio to daptomycin of 0.05:1, glycerol in an amount of 8% V/V, PEG 400 in an amount of 10% V/V, and trehalose in a molar ratio to daptomycin of 5:1, and wherein the composition has a pH of 7.

In an aspect, the aqueous pharmaceutical composition comprises daptomycin, calcium in a molar ratio of calcium to daptomycin of 1:1, at least one amino acid in a molar ratio to daptomycin of 0.05:1, glycerol in an amount of 8% V/V, PEG 400 in an amount of 10% V/V, and a sugar in a molar ratio to daptomycin of 5:1, and wherein the composition has a pH of 7.

In an aspect, the aqueous pharmaceutical composition comprises daptomycin, calcium in a molar ratio of calcium to daptomycin of 1:1 and at least one amino acid in a molar ratio to daptomycin of 0.05:1, glycerol in an amount of 8% V/V, PEG 400 in an amount of 10% V/V, a sugar in a molar ratio to daptomycin of 5:1, and a carboxylic acid in a molar ratio to daptomycin of 0.05, and wherein the composition has a pH of 7.

In an aspect, the aqueous pharmaceutical composition comprises daptomycin, calcium saccharin in a molar ratio of calcium to daptomycin of 1:1, at least one amino acid selected from L-proline and L-tyrosine in a molar ratio to daptomycin of 0.05:1, glycerol in an amount of 8% VAT, PEG 400 in an amount of 10% VAT, and a sugar selected from sucrose and trehalose in a molar ratio to daptomycin of 5:1, and wherein the composition has a pH of 7.

All of the numbers used herein are modified by the term “about.” This means that each number includes minor variations as defined ±10% of the numerical value or range in question.

As used herein, the term “pH” is defined as ±0.3 of the numerical value or range in question.

Additionally, the compositions described herein may further comprise one or more pharmaceutically acceptable excipients such as antioxidants, surfactants, complexing agents, preservatives, stabilizers, bulking agents, buffers, diluents, vehicles, solubilizers, binders, and combinations thereof. In some embodiments, the composition does not comprise a buffer.

Other objects, features and advantages will become apparent from the following detailed description and examples. It should be understood, however, that the detailed description and the examples, while indicating specific embodiments, are given by way of illustration only, and are not intended to limit the breadth or scope of the concepts in any manner.

Stable pharmaceutical compositions of daptomycin as described herein have sufficient stability to allow storage at a convenient temperature, such as from 2° C. to 8° C., for a reasonable period of time.

Disclosed herein are pharmaceutically acceptable formulations of daptomycin are stable over the course of typical storage conditions, including time periods of 7 days (1 week), 14 days (2 weeks), 30 days (1 month), 60 days (2 months), 3 months, 4 months, 180 days (6 months), 12 months (1 year), and longer, at temperatures of 2-8° C.

Furthermore, the aqueous pharmaceutical formulation of daptomycin in described herein has improved stability at room temperature conditions, i.e., at a temperature 25° C. The aqueous pharmaceutical formulations of daptomycin described herein are stable over the course of typical storage conditions, including time periods of 3 days, 4 days, 5 days, 7 days (1 week), 14 days (2 weeks), and longer at temperature of 30° C., which clearly shows their stability on the room temperature, i.e. 25° C. for the respective time periods.

The formulations comprise therapeutically effective amounts of daptomycin, wherein therapeutically effective amounts include concentrations ranging from 0.5 mg/mL to 500 mg/mL, from 2 mg/ml to 20 mg/ml, from 20 mg/mL to 400 mg/mL, from 50 mg/mL to 300 mg/mL, such as concentration of 0.5 mg/mL, 1 mg/mL, 3 mg/mL, 5 mg/mL, 8 mg/mL, 10 mg/mL, 15 mg/mL, 20 mg/mL, 25 mg/mL, 30 mg/mL, 35 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, 220 mg/mL, 240 mg/mL, 260 mg/mL, 280 mg/mL, 300 mg/mL, 350 mg/mL, 400 mg/mL, 450 mg/mL and 500 mg/mL.

The language “therapeutically effective amount” or “therapeutically effective concentrations” of the daptomycin compound, as used herein, refers to an amount of daptomycin administered to a patient sufficient to produce a therapeutic response to one or more of the symptoms of the disease being treated. Dilution prior to administration may provide a therapeutically effective amount or therapeutically effective concentration. The formulations described herein may be diluted before administration to a patient.

The formulations described herein can be further diluted with diluent(s) in order to achieve lower therapeutically effective concentrations and the “diluent(s)” of interest herein is one which is pharmaceutically acceptable; safe and non-toxic for administration to a human, and is compatible for the preparation of a diluted formulation. In some embodiments, the formulations described herein may be packaged in a vial for dilution prior to administration a patient.

Exemplary diluents include sterile water for injection, sterile saline solution and Lactated Ringer's Injection solution.

For example, in a typical preparation of diluted formulations, the appropriate volume of the aqueous formulation needed for the required therapeutically effective dose can be aseptically withdrawn and transferred into an infusion bag of a suitable diluent, such as 0.225%, 0.45% or 0.9% Sodium Chloride, or Sterile Water for Injection or Lactated Ringer's Injection and administrated to a patient via appropriate route of administration.

The aqueous formulations of daptomycin described herein are intended to be administered via injection, for example subcutaneously, intracutaneously, intravenously, intramuscularly, intraarticularly, intrasynovially, intrasternally, intrathecally, intralesionally, intracranially or via intravenous infusion.

Also within the scope are uses of pharmaceutical formulations of daptomycin, as disclosed herein, for treating infections or diseases caused by Gram positive bacteria such as complicated skin and soft-tissue infections (cSSTI), Staphylococcus aureus bloodstream infections (bacteremia), including those with right-sided infective endocarditis (RIE).

These uses comprise administering to the patient a therapeutically effective amount of formulations or administering to the patient a therapeutically effective amount of preparation prepared from a pharmaceutical formulation.

In some embodiments, the aqueous pharmaceutical formulation does not comprise an alcohol with an aromatic group, aliphatic alcohol containing only one or more primary hydroxylic groups or alcohol containing less hydroxyl groups than carbon atoms as a polar protic solvent.

In some embodiments, the formulation does not comprise at least one of: (a) a polar aprotic solvent (such as dimethylacetamide (DMA), N,N-diethylacetamide (DEA), N-ethylacetamide, N,N-dimethylproionamide, N-ethylformamide, ethyl acetate); (b) an alcohol with an aromatic group, aliphatic alcohol containing only one or more primary hydroxylic groups or alcohol containing less hydroxyl groups than carbon atoms (such as benzyl alcohol, ethanol, isobutanol, or terbutyl alcohol) as a polar protic solvent; (c) a solubilizer (such as a Kolliphor EL™ (polyethoxylated castor oil), soybean oil, polysorbate 20, polysorbate 80); and/or (d) ethylene glycol or propylene glycol.

In some embodiments, the formulation does not comprise a polar protic solvent chosen from alkyl alcohols, ethanol, benzyl alcohol, ethylene glycol, propylene glycol, butylene glycol, polysorbates, for example polysorbate 20, polysorbate 40, and polysorbate 80, cyclodextrins (such as hydroxypropyl-(1-cyclodextrin), polypropylene glycol, and polybutylene glycol, and primary amides such as niacinamide.

In some embodiments, the formulation does not comprise a polar aprotic solvent chosen from ethyl acetate, dimethyl sulfoxide (DMSO), secondary and tertiary amides, wherein secondary amides are selected from N-ethylacetamide, N-ethylformamide, and tertiary amides are selected from dimethylacetamide (DMA), N-methyl-N-vinylacetamide, N,N-dimethylpropionamide, N,N-diethylacetamide (DEA), N,N-diisopropylformamide and N,N-dimethyl formamide.

### Materials and Methods

Compositions were prepared by providing an aqueous, ready to use solution of daptomycin.

Predefined amount of calcium salt (e.g. chloride, saccharin) and different excipient(s) are dispensed. Substances are added following the predefined order of addition into the vessel containing WFI and solution is mixed until raw materials are dissolved. Daptomycin is then added into the solution and solution is mixed until daptomycin is dissolved. pH of the solution is adjusted to the predetermined pH value, using a pH adjuster comprising an acid or a base (specifically, diluted hydrochloric acid or sodium hydroxide solution depending on the direction of pH adjustment desired). Organic solvent parts of the formulation are added and homogenously mixed with the formulation and batch volume make up is performed.

Solutions were then mixed to ensure homogeneity, filtered through a 0.2 μm filter and transferred to vials.

The solution is than filled into Type I glass vials and stoppered using Type I rubber stoppers, and sealed with an overseal.

All formulations had acceptable color, clarity and no visible particles was retained.

### EXAMPLES

In the Examples and tables presented below, the following abbreviations were used:
AHD—Anhydrodaptomycin impurity
Beta—Beta (β-aspartyl) isomer
LHD—Lactone hydrolysis product
Ca²⁺—Calcium
DAP—Daptomycin
NADA—N-acetyl-D-alanine
NALA—N-acetyl-L-alanine
Ala—Alanine
Asn—Asparagine
Gln—Glutamine
Glu—Glutamic acid
Gly—Glycine
Leu—Leucine
Met—Methionine
Orn—Ornithine
Phe—Phenylalanine
Pro—Proline
Ser—Serine
Trp—Tryptophan
Tyr—Tyrosine
Val—Valine
M—Month(s)

After the aqueous compositions have been prepared and filled into vials, initial time point level of impurities was determined by HPLC and afterwards vials were loaded to stability chambers at different storage conditions, such as 5° C., 15° C., 30° C.

In order to determine formation of impurities and stability of daptomycin in formulations, vials were taken from stability chambers at various time points such as 4 days, 2 weeks, 1 month, 2 months, 4 months, 6 months, 9 months, 12 months, 14 months etc. and analyzed by HPLC.

The amount of total impurities and three structurally related compounds, lactone hydrolysis product, beta isomer of daptomycin and anhydrodaptomycin, was determined by UHPLC analysis in solutions containing daptomycin, using an Agilent 1290 ultrahigh performance liquid chromatography instrument, equipped with an ultraviolet (UV) detector. All samples were analyzed using a reverse-phase C18 column and by measuring the absorbance (area under the curve) at a wavelength of 225 nm. The content of the three structurally related compounds (impurities) is given as area % of the total area, calculated using the following equation:

$$area \% = \frac{{A}_{i}}{{A}_{tot}} \times 100 \%$$

where:

area %=area % of an individual peak

Aₜ=peak area of an individual peak

Aₜₒₜ=total sample peak area

The percent of total impurities is reported as a sum of area % of all peaks, other than the main peak (i.e. daptomycin), equal or above reporting threshold (0.05%).

### Calculation of Total Impurities

TPₘ—Value of total impurities at time point (TP) different than initial, for example: 4 days, 1 month, 2 months etc. at different storage conditions such as 30° C., 2-8° C., determined by HPLC

Δ—Calculated increase of total impurities

$$Δ Total impurities increase ( \% ) = Total impurities value at {TP}_{n} ( \% ) - Total impurities initial value ( \% )$$

Calculation of the specific impurity, i.e., anhydrodaptomycin, beta isomer impurity and lactone hydrolysis product impurity

TPₙ—Value of specific impurity at time point different than initial, for example: 4 days, 1 month, 2 months etc. at different storage conditions such as 30° C., 2-8° C., determined by HPLC

Δ—Calculated increase of the specific impurity: (%)

$$Δ specific impurity increase ( \% ) = Specific impurity value at {TP}_{n} ( \% ) - Specific impurity initial value ( \% )$$

### Example 1. Daptomycin Stability Studies in Aqueous Solutions Comprising Daptomycin in a Concentration of 50 mg/mL, Various Calcium Sources at Targeted pH 6.1

| | Molar | | | | | | | | | Total | Δ Total |
|---|---|---|---|---|---|---|---|---|---|---|---|
| Ca | ratio | Condition | Time | | Δ | | Δ | | Δ | impu- | impu- |
| source | DAP:Ca | temperature | point | AHD % | AHD % | Beta % | Beta % | LHD % | LHD % | rities % | rities % |
| / | 1:0 | 30° C. | START | 0.68 | 7.0 | 0.07 | 1.9 | 0.14 | 0.44 | 3.5 | 10.1 |
| | | | 4 days | 7.7 | | 1.9 | | 0.58 | | 13.5 | |
| | | 2-8° C. | 1 M | 2.9 | 2.3 | 0.29 | 0.22 | 0.28 | 0.14 | 6.2 | 2.8 |
| CaCl₂ | 1:1 | 30° C. | START | 0.63 | 2.5 | 0.08 | 1.3 | 0.12 | 0.43 | 3.4 | 4.7 |
| | | | 4 days | 3.2 | | 1.4 | | 0.55 | | 8.1 | |
| | | 2-8° C. | 1 M | 1.3 | 0.7 | 0.27 | 0.19 | 0.22 | 0.10 | 4.5 | 1.1 |
| | | | 12 M | 3.7 | 3.0 | 5.9 | 5.8 | 1.1 | 0.94 | 14.1 | 10.7 |
| Ca- | 1:1 | 30° C. | START | 0.61 | 2.3 | 0.11 | 1.2 | 0.26 | 0.37 | 3.0 | 4.8 |
| saccharin | | | 4 days | 2.9 | | 1.3 | | 0.63 | | 7.8 | |
| | | 2-8° C. | 1 M | 1.0 | 0.43 | 0.20 | 0.09 | 0.31 | 0.05 | 4.0 | 1.0 |
| | | | 12 M | 3.3 | 2.7 | 5.2 | 5.1 | 1.0 | 0.78 | 13.2 | 10.2 |

### Example 2a. Daptomycin Stability Studies in Aqueous Solutions Comprising Daptomycin in a Concentration of 50 mg/mL, Calcium Chloride and Various Amino Acids, at Target pH of 6.9

| | Molar ratio | | Time | | | | | | | Total | |
|---|---|---|---|---|---|---|---|---|---|---|---|
| Amino | DAP:Ca²⁺: | Condition | point/ | | Δ | | Δ | | Δ | impu- | Δ Total |
| acid | Amino acid | temperature | months | AHD % | AHD % | Beta % | Beta % | LHD % | LHD % | rities % | impurities |
| none | 1:0:0 | START | START | 0.43 | 1.97 | 0.11 | 15.89 | 0.28 | 4.92 | 3.5 | 26.8 |
| | | 2-8° C. | 12 | 2.4 | | 16.0 | | 5.2 | | 30.3 | |
| D-Ala | 1:1:0.5 | START | START | 0.47 | 0.40 | 0.11 | 5.99 | 0.71 | 2.09 | 4.0 | 9.6 |
| | | 2-8° C. | 15.5 | 0.87 | | 6.1 | | 2.8 | | 13.6 | |
| L-Ala | 1:1:0.5 | START | START | 0.47 | 0.08 | 0.15 | 4.95 | 0.26 | 2.64 | 3.7 | 8.7 |
| | | 2-8° C. | 15.5 | 0.55 | | 5.1 | | 2.9 | | 12.4 | |
| L-Ala | 1:1:2 | START | START | 0.49 | 0.16 | 0.11 | 5.49 | 0.29 | 2.71 | 3.5 | 9.4 |
| | | 2-8° C. | 15 | 0.65 | | 5.6 | | 3.0 | | 12.9 | |
| L-Asn | 1:1:0.5 | START | START | 0.49 | 0.05 | 0.15 | 4.75 | 0.33 | 2.97 | 3.8 | 8.5 |
| | | 2-8° C. | 14.5 | 0.44 | | 4.9 | | 3.3 | | 12.3 | |
| L-Gln | 1:1.0.1 | START | START | 0.45 | 0.31 | 0.11 | 5.09 | 0.28 | 2.42 | 3.6 | 8.9 |
| | | 2-8° C. | 15 | 0.76 | | 5.2 | | 2.7 | | 12.5 | |
| L-Gln | 1:1:1 | START | START | 0.41 | 0.36 | 0.11 | 5.29 | 0.42 | 2.58 | 3.6 | 9.3 |
| | | 2-8° C. | 15.5 | 0.77 | | 5.4 | | 3.0 | | 12.9 | |
| L-Glu | 1:1:0.5 | START | START | 0.45 | 0.23 | 0.18 | 5.62 | 1.3 | 2.7 | 4.8 | 10.1 |
| | | 2-8° C. | 15.5 | 0.68 | | 5.8 | | 4.0 | | 14.9 | |
| Gly | 1:1:0.1 | START | START | 0.46 | 0.24 | 0.11 | 4.79 | 0.23 | 2.37 | 3.5 | 8.4 |
| | | 2-8° C. | 14.5 | 0.70 | | 4.9 | | 2.6 | | 11.9 | |
| L-Leu | 1:1:1 | START | START | 0.45 | 0.24 | 0.13 | 4.87 | 0.29 | 2.31 | 3.6 | 8.3 |
| | | 2-8° C. | 16 | 0.69 | | 5.0 | | 2.6 | | 11.9 | |
| L-Met | 1:1.0.1 | START | START | 0.44 | 0.27 | 0.12 | 5.08 | 0.55 | 2.45 | 3.8 | 8.8 |
| | | 2-8° C. | 15 | 0.71 | | 5.2 | | 3.0 | | 12.6 | |
| L-Met | 1:1:0.5 | START | START | 0.50 | 0.30 | 0.14 | 5.66 | 0.27 | 2.53 | 3.8 | 9.5 |
| | | 2-8° C. | 15.5 | 0.80 | | 5.8 | | 2.8 | | 13.3 | |
| D-Orn | 1:1:0.1 | START | START | 0.47 | 0.14 | 0.12 | 5.08 | 0.53 | 2.47 | 3.8 | 8.8 |
| HCl | | 2-8° C. | 15 | 0.61 | | 5.2 | | 3.0 | | 12.6 | |
| L-Orn | 1:1:0.5 | START | START | 0.49 | 0.17 | 0.11 | 5.29 | 0.32 | 2.68 | 3.8 | 9.2 |
| HCl | | 2-8° C. | 15 | 0.66 | | 5.4 | | 3.0 | | 13.0 | |
| L-Phe | 1:1:0.1 | START | START | 0.49 | 0.23 | 0.10 | 4.4 | 0.25 | 1.95 | 3.5 | 7.5 |
| | | 2-8° C. | 14.5 | 0.72 | | 4.5 | | 2.2 | | 11.0 | |
| L-Pro | 1:1:2 | START | START | 0.47 | 0.21 | 0.11 | 4.89 | 0.27 | 2.43 | 3.6 | 8.5 |
| | | 2-8° C. | 16 | 0.68 | | 5.0 | | 2.7 | | 12.1 | |
| L-Ser | 1:1:10 | START | START | 0.50 | 0.07 | 0.08 | 5.02 | 0.41 | 3.09 | 3.7 | 10.1 |
| | | 2-8° C. | 16 | 0.57 | | 5.1 | | 3.5 | | 13.8 | |
| L-Trp | 1:1:0.1 | START | START | 0.47 | 0.32 | 0.13 | 4.87 | 0.53 | 2.07 | 3.8 | 8.3 |
| | | 2-8° C. | 14.5 | 0.79 | | 5.0 | | 2.6 | | 12.1 | |
| L-Tyr | 1:1:0.05 | START | START | 0.44 | 0.21 | 0.13 | 4.27 | 1.1 | 2.1 | 4.3 | 7.6 |
| | | 2-8° C. | 14.5 | 0.65 | | 4.4 | | 3.2 | | 11.9 | |
| L-Val | 1:1:0.1 | START | START | 0.47 | 0.31 | 0.11 | 5.09 | 0.31 | 2.19 | 3.6 | 8.8 |
| | | 2-8° C. | 14.5 | 0.78 | | 5.2 | | 2.5 | | 12.4 | |
| L-Val | 1:1:2 | START | START | 0.46 | 0.21 | 0.12 | 5.68 | 1.2 | 2.7 | 4.6 | 9.7 |
| | | 2-8° C. | 15 | 0.67 | | 5.8 | | 3.9 | | 14.3 | |
| D-Val | 1:1:0.1 | START | START | 0.47 | 0.29 | 0.12 | 5.78 | 0.47 | 2.13 | 3.7 | 9.5 |
| | | 2-8° C. | 15 | 0.76 | | 5.9 | | 2.6 | | 13.2 | |

### Example 2b. Daptomycin Stability Studies in Aqueous Solutions Comprising Daptomycin in a Concentration of 50 mg/mL, Calcium Chloride 69£6ZZ/OZOZ OM and Chemically Modified Amino Acids at Target pH of 6.9

| | Molar ratio | | Time | | | | | | | Total | Δ Total |
|---|---|---|---|---|---|---|---|---|---|---|---|
| | DAP:Ca²⁺: | Condition | point/ | | Δ | | Δ | | Δ | impu- | impu- |
| Amino acid | Amino acid | temperature | month | AHD % | AHD % | Beta % | Beta % | LHD % | LHD % | rities % | rities % |
| N-formyl-Gly | 1:1:0.1 | START | START | 0.44 | 0.26 | 0.12 | 4.78 | 0.42 | 2.38 | 3.7 | 8.3 |
| | | 2-8° C. | 14.5 | 0.70 | | 4.9 | | 2.8 | | 12.0 | |
| NADA | 1:1:0.1 | START | START | 0.46 | 0.26 | 0.13 | 5.07 | 1.4 | 2.0 | 4.6 | 8.7 |
| | | 2-8° C. | 14.5 | 0.72 | | 5.2 | | 3.4 | | 13.3 | |
| NALA | 1:1:0.1 | START | START | 0.50 | 0.16 | 0.12 | 4.88 | 0.34 | 2.26 | 3.6 | 8.2 |
| | | 2-8° C. | 15 | 0.66 | | 5.0 | | 2.6 | | 11.8 | |

### Example 2c. Daptomycin Stability Studies in Aqueous Solutions Comprising Daptomycin in a Concentration of 50 mg/mL, Calcium Chloride and Mixture of Two Amino Acids at Targeted pH of 6.9

| | Molar ratio | | Time | | | | | | | Total | Δ Total |
|---|---|---|---|---|---|---|---|---|---|---|---|
| | DAP:Ca²⁺: | Condition | point/ | | Δ | | Δ | | Δ | impu- | impu- |
| Amino acid | Amino acid | temperature | months | AHD % | AHD % | Beta % | Beta % | LHD % | LHD % | rities % | rities |
| Gly, L-Met | 1:1:0.1:0.1 | START | START | 0.46 | 0.17 | 0.11 | 4.89 | 0.32 | 2.38 | 3.6 | 8.5 |
| | | 2-8° C. | 15 | 0.63 | | 5.0 | | 2.7 | | 12.1 | |
| L-Val, L-Met | 1:1:0.1:0.1 | START | START | 0.46 | 0.24 | 0.10 | 5.1 | 0.29 | 2.41 | 3.4 | 8.9 |
| | | 2-8° C. | 15 | 0.70 | | 5.2 | | 2.7 | | 12.3 | |

### Example 3. Daptomycin Stability Studies in Aqueous Solutions Comprising Daptomycin in a Concentration of 50 mg/mL, Calcium Chloride and Excipients Selected from Betaine, Taurine, Nicotinamide and Sugars, at pH of 6.0-7.0

| | Molar ratio | | | Time | | | | | | | Total | Δ Total |
|---|---|---|---|---|---|---|---|---|---|---|---|---|
| | DAP:Ca²⁺: | | Condition | point/ | | Δ | | Δ | | Δ | impu- | impu- |
| Excipient | Excipient | pH | temperature | months | AHD % | AHD % | Beta % | Beta % | LHD % | LHD % | rities % | rities % |
| betaine | 1:1:2 | 7.0 | START | START | 0.46 | 0.05 | 0.11 | 3.69 | 0.33 | 2.27 | 3.5 | 6.8 |
| | | 6.9 | 2-8° C. | 12 | 0.51 | | 3.8 | | 2.6 | | 10.3 | |
| taurine | 1:1:0.1 | 7.1 | START | START | 0.45 | 0.21 | 0.13 | 5.27 | 0.38 | 2.62 | 3.7 | 9.1 |
| | | 6.8 | 2-8° C. | 15.5 | 0.66 | | 5.4 | | 3.0 | | 12.8 | |
| taurine | 1:1:2 | 6.9 | START | START | 0.50 | 0.30 | 0.09 | 5.81 | 0.21 | 2.49 | 3.5 | 9.6 |
| | | 6.7 | 2-8° C. | 14 | 0.80 | | 5.9 | | 2.7 | | 13.1 | |
| nicotinamide | 1:1:0.1 | 6.8 | START | START | 0.46 | 0.34 | 0.13 | 4.67 | 0.49 | 1.91 | 3.9 | 7.8 |
| | | 6.7 | 2-8° C. | 14.5 | 0.80 | | 4.8 | | 2.4 | | 11.7 | |
| nicotinamide | 1:1:0.5 | 6.9 | START | START | 0.48 | 0.25 | 0.12 | 4.58 | 0.36 | 2.24 | 3.6 | 8.0 |
| | | 6.7 | 2-8° C. | 14 | 0.73 | | 4.7 | | 2.6 | | 11.6 | |
| nicotinamide | 1:1:2 | 6.9 | START | START | 0.45 | 0.27 | 0.12 | 5.28 | 1.1 | 2.6 | 4.3 | 9.4 |
| | | 6.8 | 2-8° C. | 15 | 0.72 | | 5.4 | | 3.7 | | 13.7 | |
| Sucrose | 1:1:1 | 7.0 | START | START | 0.48 | 0.6 | 0.11 | 4.49 | 0.52 | 2.18 | 3.7 | 7.9 |
| | | 6.7 | 2-8° C. | 13 | 0.74 | | 4.6 | | 2.7 | | 11.6 | |
| Sucrose | 1:1:20 | 6.5 | START | START | 0.56 | 0.58 | 0.10 | 4.1 | 0.50 | 1.2 | 5.0 | 5.5 |
| | | 6.5 | 2-8° C. | 11 | 1.14 | | 4.2 | | 1.7 | | 10.5 | |
| Sucrose | 1:2:5 | 6.0 | START | START | 0.53 | 0.77 | 0.07 | 1.33 | 0.18 | 0.31 | 3.5 | 2.5 |
| | | 6.0 | 2-8° C. | 6 | 1.3 | | 1.4 | | 0.49 | | 6.0 | |
| Sucrose | 1:2.7:8 | 5.9 | START | START | 0.52 | 0.38 | 0.06 | 0.80 | 0.15 | 0.20 | 3.4 | 1.5 |
| | | 6.0 | 2-8° C. | 4 | 0.90 | | 0.86 | | 0.35 | | 4.9 | |
| Trehalose | 1:1:1 | 7.0 | START | START | 0.45 | 0.10 | 0.13 | 3.87 | 0.60 | 2.0 | 3.8 | 6.8 |
| | | 6.8 | 2-8° C. | 12 | 0.55 | | 4.0 | | 2.6 | | 10.6 | |
| Fructose | 1:1:5 | 7.0 | START | START | 0.48 | 0.19 | 0.09 | 4.11 | 0.47 | 2.03 | 3.7 | 7.3 |
| | | 6.8 | 2-8° C. | 12 | 0.67 | | 4.2 | | 2.5 | | 11.0 | |

### Example 4. Daptomycin Stability Studies in Aqueous Solutions Comprising Daptomycin in a Concentration of 50 mg/mL, Calcium Chloride and Excipients Selected from Amino Acids and Sugars, at pH of 6.0

| | Molar ratio | | Time | | | | | | | Total | Δ Total |
|---|---|---|---|---|---|---|---|---|---|---|---|
| | DAP:Ca²⁺: | Condition | point/ | | Δ | | Δ | | Δ | impu- | impu- |
| Excipient | Excipient | temperature | months | AHD % | AHD % | Beta % | Beta % | LHD % | LHD % | rities % | rities % |
| / | 1:2:0 | START | START | 0.49 | 1.2 | 0.12 | 1.5 | 0.17 | 0.29 | 3.4 | 3.1 |
| | | 2-8° C. | 6 M | 1.7 | | 1.6 | | 0.46 | | 6.5 | |
| L-phenylalanine | 1:2:0.1 | START | START | 0.45 | 0.68 | 0.10 | 1.4 | 0.10 | 0.39 | 3.3 | 2.6 |
| | | 2-8° C. | 6 M | 1.1 | | 1.5 | | 0.49 | | 5.9 | |
| L-tryptophan | 1:2:0.1 | START | START | 0.48 | 0.86 | 0.06 | 1.5 | 0.09 | 0.35 | 3.3 | 2.8 |
| | | 2-8° C. | 6 M | 1.3 | | 1.6 | | 0.44 | | 6.2 | |
| L-proline | 1:2:0.1 | START | START | 0.48 | 0.82 | 0.07 | 1.5 | 0.08 | 0.37 | 3.3 | 2.9 |
| | | 2-8° C. | 6 M | 1.3 | | 1.6 | | 0.45 | | 6.1 | |
| D(+)-trehalose | 1:2:10 | START | START | 0.51 | 0.61 | 0.07 | 1.4 | 0.12 | 0.36 | 3.5 | 2.5 |
| | | 2-8° C. | 6 M | 1.1 | | 1.5 | | 0.48 | | 5.9 | |
| D(+)-raffinose | 1:2:4 | START | START | 0.50 | 0.64 | 0.07 | 1.4 | 0.11 | 0.33 | 3.3 | 2.5 |
| | | 2-8° C. | 6 M | 1.1 | | 1.5 | | 0.44 | | 5.8 | |
| Meglumine | 1:2:0.1 | START | START | 0.48 | 0.75 | 0.09 | 1.5 | 0.21 | 0.36 | 3.5 | 2.8 |
| | | 2-8° C. | 6 M | 1.2 | | 1.6 | | 0.57 | | 6.2 | |

### Example 5. Daptomycin Stability Studies in Aqueous Solutions Comprising Daptomycin in a Concentration of 50 mg/mL, Calcium, Amino Acid (s), Organic Solvent(s), Sugar(s) and Sucralose at Targeted pH of 6.3

| | | | | | | | | | | Δ |
|---|---|---|---|---|---|---|---|---|---|---|
| Excipients (molar ratio of | | | | | | | | | Total | Total |
| excipients to daptomycin | Condition | Time | | Δ | | Δ | | Δ | impu- | impu- |
| molar ratio 1) | temperature | point | AHD % | AHD % | Beta % | Beta % | LHD % | LHD % | rities | rities % |
| CaCl2 (Ca²⁺ molar ratio:1) | START | START | 0.63 | 1.9 | 0.08 | 1.2 | 0.12 | 0.31 | 3.4 | 3.6 |
| | 2-8° C. | 4 M | 2.5 | | 1.3 | | 0.43 | | 7.0 | |
| CaCl2 1, L-Pro 0.05, L-Tyr | START | START | 0.64 | 1.6 | 0.13 | 1.1 | 0.20 | 0.33 | 3.8 | 3.6 |
| 0.025, sucrose 5, sucralose | 2-8° C. | 4 M | 2.2 | | 1.2 | | 0.53 | | 7.4 | |
| 0.25, glycerol 7.9% V/V, | | | | | | | | | | |
| PEG400 sup ref 10% V/V | | | | | | | | | | |
| CaCl2 1, L-Pro 0.1, L-Tyr | START | START | 0.65 | 1.2 | 0.12 | 1.0 | 0.19 | 0.29 | 3.6 | 3.2 |
| 0.05, sucrose 5, sucralose | 2-8° C. | 4 M | 1.8 | | 1.1 | | 0.48 | | 6.7 | |
| 0.25, glycerol 7.9% V/V, | | | | | | | | | | |
| PEG400 sup ref 10% V/V | | | | | | | | | | |
| CaCl2 1, L-Pro 0.1, L-Tyr | START | START | 0.69 | 1.3 | 0.13 | 1.0 | 0.34 | 0.25 | 4.0 | 3.0 |
| 0.025, sucrose 9.4, sucralose | 2-8° C. | 4 M | 2.0 | | 1.1 | | 0.59 | | 7.0 | |
| 0.25, glycerol 7.9% V/V, | | | | | | | | | | |
| PEG400 sup ref 10% V/V | | | | | | | | | | |
| CaCl2 1, L-Pro 0.05, L-Tyr | START | START | 0.70 | 1.0 | 0.09 | 0.87 | 0.24 | 0.25 | 3.7 | 3.0 |
| 0.05, sucrose 9.4, raffinose 4, | 2-8° C. | 4 M | 1.7 | | 0.96 | | 0.49 | | 6.7 | |
| sucralose 3.4, glycerol 7.9% | | | | | | | | | | |
| V/V, PEG400 sup ref 10% | | | | | | | | | | |
| V/V | | | | | | | | | | |
| CaCl2 1, L-Pro 0.1, L-Tyr | START | START | 0.67 | 1.1 | 0.10 | 0.84 | 0.21 | 0.24 | 3.8 | 2.8 |
| 0.05, sucrose 5, raffinose 4, | 2-8° C. | 4 M | 1.8 | | 0.94 | | 0.45 | | 6.7 | |
| sucralose 3.4, glycerol 7.9% | | | | | | | | | | |
| V/V, PEG400 sup ref 10% | | | | | | | | | | |
| V/V | | | | | | | | | | |
| CaCl2 1, L-Pro 0.1, L-Tyr | START | START | 0.71 | 1.1 | 0.12 | 0.79 | 0.29 | 0.23 | 3.7 | 2.9 |
| 0.025, sucrose 9.4, raffinose | 2-8° C. | 4 M | 1.8 | | 0.91 | | 0.52 | | 6.6 | |
| 4, sucralose 3.4, glycerol | | | | | | | | | | |
| 7.9% V/V, PEG400 sup ref | | | | | | | | | | |
| 10% V/V | | | | | | | | | | |

### Example 6. Daptomycin Stability Studies in Aqueous Solutions Comprising Daptomycin in a Concentration of 50 mg/mL, Calcium, Amino Acid (s), Organic Solvent(s), Sugar(s) and Carboxylic Acids at Various Targeted pH 7.2

| | | | | | | | | | Total | Δ Total |
|---|---|---|---|---|---|---|---|---|---|---|
| Excipients (molar ratio of excipients to | Condition | Time | | Δ | | Δ | | Δ | impu- | impu- |
| daptomycin 1) | temperature | point | AHD % | AHD % | Beta % | Beta % | LHD % | LHD % | rities % | rities % |
| CaCl2 (Ca²⁺ molar ratio:1) | START | START | 0.58 | −0.16 | 0.10 | 0.38 | 0.16 | 0.23 | 3.2 | 0.73 |
| | 2-8° C. | 1 M | 0.42 | −0.19 | 0.48 | 0.69 | 0.39 | 0.42 | 3.9 | 1.3 |
| | 30° C. | 2 M | 0.39 | −0.01 | 0.79 | 1.7 | 0.58 | 1.5 | 4.5 | 3.9 |
| | | 4 days | 0.57 | | 1.8 | | 1.7 | | 7.1 | |
| Ca(saccharin)2 (Ca²⁺ molar ratio:1), L-Pro 0.05, | START | START | 0.47 | −0.09 | 0.05 | 0.30 | 0.13 | 0.17 | 3.2 | 0.38 |
| L-Tyr 0.05, sucrose 5, glycerol 8% V/V, PEG400 | 2-8° C. | 1 M | 0.38 | −0.05 | 0.35 | 0.55 | 0.30 | 0.32 | 3.6 | 1.0 |
| 10% V/V | 30° C. | 2 M | 0.42 | 0.19 | 0.60 | 1.5 | 0.45 | 1.3 | 4.2 | 3.8 |
| | | 4 days | 0.66 | | 1.6 | | 1.4 | | 7.0 | |
| CaCl2 (Ca²⁺ molar ratio:1), L-Pro 0.05, L-Tyr | START | START | 0.46 | −0.05 | 0.07 | 0.28 | 0.27 | 0.19 | 3.4 | 0.41 |
| 0.05, sucrose 5, glycerol 8% V/V, PEG400 10% | 2-8° C. | 1 M | 0.41 | 0.60 | 0.35 | 0.58 | 0.46 | 4.2 | 3.8 | |
| V/V | 30° C. | 2 M | 0.45 | −0.01 | 0.27 | 0.53 | 1.6 | 0.31 | 1.2 | 0.81 |
| | | 4 days | 0.73 | | 1.6 | | 1.5 | | 7.1 | 3.7 |
| Ca(saccharin)2 (Ca²⁺ molar ratio:1), L-Tyr 0.05, | START | START | 0.46 | −0.05 | 0.08 | 0.30 | 0.15 | 0.13 | 3.3 | 0.39 |
| sucrose 5, glycerol 8% V/V, PEG400 10% V/V | 2-8° C. | 1 M | 0.41 | 0.17 | 0.38 | 1.5 | 0.28 | 1.3 | 3.7 | 3.5 |
| | 30° C. | 4 days | 0.63 | | 1.6 | | 1.4 | | 6.8 | |
| Ca(saccharin)2 (Ca²⁺ molar ratio:1), L-Tyr 0.05, | START | START | 0.47 | −0.15 | 0.08 | 0.58 | 0.12 | 0.37 | 3.2 | 0.92 |
| Na-L-lactate 0.05, sucrose 5, glycerol 8% V/V, | 2-8° C. | 2 M | 0.32 | 0.05 | 0.66 | 1.5 | 0.49 | 1.4 | 4.1 | 3.5 |
| PEG400 10% V/V | 30° C. | 4 days | 0.52 | | 1.6 | | 1.5 | | 6.6 | |
| Ca(saccharin)2 (Ca²⁺ molar ratio:1), L-Tyr 0.05, | START | START | 0.45 | −0.13 | 0.08 | 0.57 | 0.11 | 0.37 | 3.2 | 0.83 |
| Na-L-lactate 0.05, glycerol 8% V/V, PEG400 | 2-8° C. | 2 M | 0.32 | 0.11 | 0.65 | 1.5 | 0.48 | 1.4 | 4.0 | 3.6 |
| 10% V/V | 30° C. | 4 days | 0.56 | | 1.6 | | 1.5 | | 6.8 | |
| Ca(saccharin)2 (Ca²⁺ molar ratio:1), L-Tyr 0.05, | START | START | 0.46 | −0.04 | 0.07 | 0.54 | 0.25 | 0.31 | 3.3 | 0.94 |
| Na-gluconate 0.05, glycerol 8% V/V, PEG400 | 2-8° C. | 2 M | 0.42 | 0.19 | 0.61 | 1.5 | 0.56 | 1.2 | 4.2 | 3.5 |
| 10% V/V | 30° C. | 4 days | 0.65 | | 1.6 | | 1.40 | | 6.80 | |
| Ca(saccharin)2 (Ca²⁺ molar ratio:1), L-Tyr 0.05, | START | START | 0.49 | 0.04 | 0.07 | 0.25 | 0.09 | 0.14 | 3.3 | 0.56 |
| Na-L-lactate 0.1, sucrose 5, glycerol 8% V/V, | 2-8° C. | 1 M | 0.53 | 0.39 | 0.32 | 1.4 | 0.23 | 0.94 | 3.8 | 3.4 |
| PEG400 10% V/V | 30° C. | 4 days | 0.88 | | 1.5 | | 1.0 | | 6.7 | |
| Ca(saccharin)2 (Ca²⁺ molar ratio:1), L-Tyr 0.05, | START | START | 0.43 | 0.07 | 0.05 | 0.22 | 0.10 | 0.11 | 3.0 | 0.40 |
| D(+)trehalose 5, sucrose 5, glycerol 8% V/V, | 2-8° C. | 1 M | 0.50 | 0.43 | 0.27 | 1.3 | 0.21 | 0.86 | 3.4 | 3.2 |
| PEG400 10% V/V | 30° C. | 4 days | 0.86 | | 1.4 | | 0.96 | | 6.2 | |
| CaCl2 (Ca²⁺ molar ratio:1), L-Tyr 0.05, | START | START | 0.44 | 0.11 | 0.05 | 0.22 | 0.24 | 0.11 | 3.3 | 0.30 |
| D(+)trehalose 5, sucrose 5, glycerol 8% V/V, | 2-8° C. | 1 M | 0.55 | 0.61 | 0.27 | 1.4 | 0.35 | 0.84 | 3.6 | 3.5 |
| PEG400 10% V/V | 30° C. | 4 days | 1.1 | | 1.4 | | 1.1 | | 6.8 | |
| Ca(saccharin)2 (Ca²⁺ molar ratio:1), L-Tyr 0.05, | START | START | 0.45 | −0.02 | 0.10 | 0.28 | 0.12 | 0.15 | 3.5 | 0.3 |
| Na-L-lactate 0.05, D(+)trehalose 5, glycerol 8% | 2-8° C. | 1 M | 0.43 | 0.26 | 0.38 | 1.4 | 0.27 | 1.1 | 3.8 | 2.9 |
| V/V PEG400 10% V/V | 30° C. | 4 days | 0.71 | | 1.5 | | 1.2 | | 6.4 | |

### Example 7. Daptomycin Stability Studies in Aqueous Solutions Comprising Daptomycin in a Concentration of 50 mg/mL, Calcium Chloride and One or Two Organic Solvents at Targeted pH of 6.0

| | Molar | | | | | | | | | Total | Δ Total |
|---|---|---|---|---|---|---|---|---|---|---|---|
| | ratio | Condition | Time | | Δ | | Δ | | Δ | impu- | impu- |
| Component 1 | DAP:Ca²⁺ | temperature | point | AHD | AHD | Beta | Beta | LHP | LHP | rities | rities |
| / | 1:0 | START | START | 0.68 | 7.0 | 0.07 | 1.9 | 0.14 | 0.44 | 3.5 | 10.1 |
| | | 30° C. | 4 days | 7.7 | | 1.9 | | 0.58 | | 13.5 | |
| glycerol 7.9% (V/V) | 1:1 | START | START | 0.61 | 2.3 | 0.09 | 1.2 | 0.19 | 0.43 | 3.4 | 4.4 |
| | | 30° C. | 4 days | 2.9 | | 1.3 | | 0.62 | | 7.7 | |
| glycerol 7.9% (V/V)), | 1:1 | START | START | 0.64 | 2.2 | 0.09 | 1.2 | 0.15 | 0.42 | 3.4 | 4.6 |
| PEG400 10% (V/V) | | 30° C. | 4 days | 2.8 | | 1.3 | | 0.57 | | 8.0 | |
| glycerol 31.9%(V/V) | 1:1 | START | START | 0.58 | 2.4 | 0.08 | 0.92 | 0.12 | 0.31 | 3.3 | 4.2 |
| | | 30° C. | 4 days | 3.0 | | 1.0 | | 0.43 | | 7.5 | |

### Example 8. Daptomycin Stability Studies in Aqueous Solutions Comprising Daptomycin in a Concentration of 50 mg/mL, Calcium Chloride and One or Two Organic Solvents at Targeted pH of 7.0

| | Molar | | | | | | | | | | |
|---|---|---|---|---|---|---|---|---|---|---|---|
| | ratio | Condition | Time | | Δ | | Δ | | Δ | Total | Δ Total |
| Components | DAP:Ca²⁺ | temperature | point | AHD | AHD | Beta | Beta | LHP | LHP | impurities | impurities |
| / | 1:0 | START | START | 0.49 | 1.3 | 0.07 | 2.1 | 0.15 | 1 | 3.4 | 4.8 |
| | | 30° C. | 4 d | 1.8 | | 2.1 | | 1.2 | | 8.2 | |
| glycerol 4.0% (V/V), PEG400 | 1:1 | START | START | 0.43 | 0.53 | 0.12 | 1.48 | 0.16 | 0.95 | 3.76 | 3.56 |
| 13% (V/V) | | 30° C. | 4 d | 0.96 | | 1.6 | | 1.11 | | 7.32 | |
| glycerol 4.0% (V/V), PEG400 | 1:1 | START | START | 0.45 | 0.51 | 0.12 | 1.49 | 0.18 | 0.93 | 3.82 | 3.43 |
| 6% (V/V) | | 30° C. | 4 d | 0.96 | | 1.61 | | 1.11 | | 7.25 | |
| glycerol 2.4% (V/V), PEG400 | 1:1 | START | START | 0.48 | 0.4 | 0.1 | 1.51 | 0.13 | 0.98 | 3.8 | 3.4 |
| 3% (V/V) | | 30° C. | 4 d | 0.88 | | 1.61 | | 1.11 | | 7.2 | |
| PEG400 10% (V/V) | 1:1 | START | START | 0.45 | 0.46 | 0.12 | 1.48 | 0.15 | 0.95 | 3.8 | 3.44 |
| | | 30° C. | 4 d | 0.91 | | 1.6 | | 1.1 | | 7.24 | |

### Example 9

The following numbered items represent embodiments of aqueous pharmaceutical formulations comprising daptomycin.

Item 1. An aqueous pharmaceutical formulation comprising daptomycin, calcium, and at least one excipient.

Item 2. The aqueous pharmaceutical formulation of item 1, wherein the at least one excipient does not comprise a buffering agent selected from carbonate buffer, citrate buffer, phosphate buffer, ADA, ACES, MES, TRIS, PIPES, MOPS, HEPES.

Item 3. The aqueous pharmaceutical formulation of any one of items from 1-2, wherein the at least one excipient comprises at least one of PEG and/or glycerol.

Item 4. The aqueous pharmaceutical formulation according to item 1, wherein the pH range of the formulation is from 5.5 to 7.5.

Item 5. The aqueous pharmaceutical formulation according to any one of items from 1-4, wherein the calcium is in the form of calcium chloride (CaCl2), Ca-α-D-heptagluconate, calcium saccharin, calcium lactate, or calcium acetate.

Item 6. The aqueous pharmaceutical formulation according to any one of items from 1-5, wherein the calcium is in the form of calcium chloride.

Item 7. The aqueous pharmaceutical formulation according to any one of items 1-5, wherein the calcium is in the form of calcium saccharin.

Item 8. The aqueous pharmaceutical formulation of any of items from 1 to 6, wherein the calcium is present in a molar ratio to daptomycin of 0.1:1 to 2:1.

Item 9. The aqueous pharmaceutical formulation of item 6, wherein the calcium is present in a molar ratio to daptomycin of 0.1:1 to 1:1.

Item 10. The aqueous pharmaceutical formulation of any one of items from 1-9, wherein the formulation does not comprise an alcohol with an aromatic group, aliphatic alcohol containing only one or more primary hydroxylic groups or alcohol containing less hydroxyl groups than carbon atoms as a polar protic solvent.

Item 11. The aqueous pharmaceutical formulation of any one of items from 1-10, wherein the formulation does not comprise at least one of:
a) a polar aprotic solvent (such as dimethylacetamide (DMA), N,N-diethylacetamide (DEA), N-ethylacetamide, N,N-dimethylproionamide, N-ethylformamide, ethyl acetate);
b) an alcohol with an aromatic group, aliphatic alcohol containing only one or more primary hydroxylic groups or alcohol containing less hydroxyl groups than carbon atoms (such as benzyl alcohol, ethanol, isobutanol, or terbutyl alcohol) as a polar protic solvent;
c) a solubilizer (such as a Kolliphor EL™ (polyethoxylated castor oil), soybean oil, polysorbate 20, polysorbate 80); and/or
d) ethylene glycol or propylene glycol.

Item 12. The aqueous pharmaceutical formulation of any one of items from 1-11, wherein the formulation does not comprise a polar protic solvent chosen from alkyl alcohols, ethanol, benzyl alcohol, ethylene glycol, propylene glycol, butylene glycol, polysorbates, for example polysorbate 20, polysorbate 40, and polysorbate 80, cyclodextrins (such as hydroxypropyl-(1-cyclodextrin), polypropylene glycol, and polybutylene glycol, and primary amides such as niacinamide.

Item 13. The aqueous pharmaceutical formulation of any one of items from 1-12, wherein the formulation does not comprise a polar aprotic solvent chosen from ethyl acetate, dimethyl sulfoxide (DMSO), secondary and tertiary amides, wherein secondary amides are selected from N-ethylacetamide, N-ethylformamide, and tertiary amides are selected from dimethylacetamide (DMA), N-methyl-N-vinylacetamide, N,N-dimethylpropionamide, N,N-diethylacetamide (DEA), N,N-diisopropylformamide and N,N-dimethyl formamide.

Item 14. The aqueous pharmaceutical formulation according to any of items from 1-13, wherein at least one excipient is selected from amino acid, sugar, sugar derivatives, saccharin, organic acids, organic solvents, betaine, taurine, nicotinamide or their pharmaceutically acceptable salts or derivatives thereof.

Item 15. The aqueous pharmaceutical formulation according to any of items from 1-14, wherein at least one excipient is selected from amino acid.

Item 16. The aqueous pharmaceutical formulation according to any of items from 1-15, wherein at least one amino acid is selected from alanine, asparagine, aspartic acid, glutamine, glutamic acid, glycine, leucine, methionine, ornithine, phenylalanine, proline, serine, tryptophan, tyrosine, valine or its pharmaceutically acceptable salt or derivative thereof.

Item 17. The aqueous pharmaceutical formulation according to any of items from 1-16, wherein the formulation comprises two or more amino acids or their pharmaceutically acceptable salts or derivatives thereof.

Item 18. The aqueous pharmaceutical formulation according to items from 15-17, wherein the molar ratio of daptomycin to each of the amino acids is from 1:0.01 to 1:10.

Item 19. The aqueous pharmaceutical formulation according to any preceding items, wherein at least one excipient is selected from organic solvents, such as alkyl alcohols, ethanol, benzyl alcohol, ethylene glycol, propylene glycol, butylene glycol, glycerol, polysorbates, for example polysorbate 20, polysorbate 40, and polysorbate 80, polyalkylene glycols, such as polyethylene glycol (PEG), polyethylene glycol 200 (PEG 200), polyethylene glycol 300 (PEG 300), polyethylene glycol 400 (PEG 400), polyethylene glycol 600 (PEG 600), polypropylene glycol, povidone and polybutylene glycol, and primary amides such as niacinamide.

Item 20. The aqueous pharmaceutical formulation according to item 19, wherein the organic solvent is glycerol.

Item 21. The aqueous pharmaceutical formulation according to item 19, wherein the organic solvent is polyethylene glycol 400 (PEG 400).

Item 22. The aqueous pharmaceutical formulation according to any one of items from 19-21, wherein the formulation comprises two or more organic solvents.

Item 23. The aqueous pharmaceutical formulation according to any one of items from 19-22, wherein formulation comprises glycerol and PEG 400.

Item 24. The aqueous pharmaceutical formulation according to any one of items from 19-23, wherein the formulation comprises each organic solvent in the amount of 20% V/V or less.

Item 25. The aqueous pharmaceutical formulation according to any one of items from 19-24, wherein the organic solvent is glycerol which is comprised in the formulation in the amount of 10% V/V or less.

Item 26. The aqueous pharmaceutical formulation according to any one of items from 19-24, wherein the organic solvent is glycerol which is comprised in the formulation in the amount of 5% V/V or less.

Item 27. The aqueous pharmaceutical formulation according to any one of items from 19-24, wherein the organic solvent is PEG 400 which is comprised in the formulation in the amount of 10% V/V or less.

Item 28. The aqueous pharmaceutical formulation according to any one of items from 19-24, wherein the organic solvent is PEG 400 which is comprised in the formulation in the amount of 5% V/V or less.

Item 29. The aqueous pharmaceutical formulation according to any of the preceding items, wherein at least one excipient is selected from saccharin, its pharmaceutically acceptable salts or derivatives thereof.

Item 30. The aqueous pharmaceutical formulation according to item 27, wherein molar ratio of daptomycin to saccharin is from 1:0.1 to 1:3.

Item 31. The aqueous pharmaceutical formulation according to any of the preceding items, wherein at least one excipient is selected from carboxylic acids, their salts or derivatives thereof.

Item 32. The aqueous pharmaceutical formulation according to item 29, wherein carboxylic acid is selected from lactic, citric, succinic and gluconic acids.

Item 33. The aqueous pharmaceutical formulation according to item 29 or 30, wherein molar ratio of daptomycin to each of selected carboxylic acid is from 1:0.05 to 1:1.

Item 34. The aqueous pharmaceutical formulation according to any of the preceding items, wherein at least one excipient is selected from sugar derivatives.

Item 35. The aqueous pharmaceutical formulation according to item 32, wherein the sugar derivative is sucralose.

Item 36. The aqueous pharmaceutical formulation according to item 33, wherein molar ratio of daptomycin to sucralose is from 1:0.05 to 1:10.

Item 37. The aqueous pharmaceutical formulation to any of the preceding items, wherein the daptomycin is at a concentration of from 0.5 mg/mL to 500 mg/mL.

Item 38. The aqueous pharmaceutical formulation of any one of items 1-35, wherein the daptomycin is at a concentration of from 2 mg/ml to 20 mg/ml.

Item 39. The aqueous pharmaceutical formulation of any one of items 1-36, wherein the daptomycin is at a concentration of 50 mg/ml.

Item 40. The aqueous pharmaceutical formulation according to any one of items from 4-39, wherein the pH is 7.

Item 41. An aqueous pharmaceutical formulation comprising 50 mg/ml of daptomycin, wherein the pharmaceutical formulation comprises daptomycin, calcium and PEG 400; wherein the molar ratio of daptomycin to calcium is 1:1, PEG 400 is comprised in concentration of 10% V/V or less and wherein the pH of the formulation is 7.

Item 42. An aqueous pharmaceutical formulation comprising 50 mg/ml of daptomycin, wherein the pharmaceutical formulation comprises daptomycin, calcium and PEG 400; wherein the molar ratio of daptomycin to calcium is 1:1, PEG 400 is comprised in concentration of 5% V/V or less and wherein the pH of the formulation is 7.

Item 43. An aqueous pharmaceutical formulation comprising 50 mg/ml of daptomycin, wherein the pharmaceutical formulation comprises daptomycin, calcium, glycerol and PEG 400; wherein the molar ratio of daptomycin to calcium is 1:1, glycerol is comprised in concentration of 5% V/V or less, PEG 400 is comprised in concentration of 5% V/V or less and wherein the pH of the formulation is 7.

Item 44. The aqueous pharmaceutical formulation according to any one of items 1-38, wherein the aqueous pharmaceutical formulation comprises at least 50% water V/V.

Item 45. The aqueous pharmaceutical formulation according to any one of items 1-39, wherein the aqueous pharmaceutical formulation comprises more than 50% water V/V.

Item 46. The aqueous pharmaceutical formulation according to any one of items 1-40, wherein the aqueous pharmaceutical formulation comprises at least 60% water V/V.

Item 47. The aqueous pharmaceutical formulation according to any one of items 1-41, wherein the aqueous pharmaceutical formulation comprises at least 50%, 60%, 70%, 80%, 85%, 90%, 95%, 98%, or 99% water VN.

Item 48. The aqueous pharmaceutical formulation of any one of items from 1-42, wherein the formulation is packaged in a vial for dilution prior to administration a patient.

Item 49. The aqueous pharmaceutical formulation according to any one of items 1-42, wherein the aqueous pharmaceutical formulation is stable for at least 4 days at temperatures of 30° C.

Item 50. The aqueous pharmaceutical formulation according to any of the preceding items, for use in treatment of microbial infections caused by Gram positive bacteria.

Item 51. The aqueous pharmaceutical formulation according to item 46, for use in treatment of skin and soft-tissue infections (cSSTI) or Staphylococcus aureus bloodstream infections (bacteremia).

Item 52. The process for manufacturing aqueous pharmaceutical formulations according to any of the preceding items, comprising steps of mixing of daptomycin, calcium and at least one excipient into solution, adjusting the pH of such solution to pH from 5.5 to 7.5 with a suitable pH adjusting agent.

Item 53. A method of treating a patient with a microbial infection comprising administering the aqueous pharmaceutical formulation according to any one of items 1-47, optionally diluting the pharmaceutical formulation before administering it to the patient.

Item 54. The method of treating a patient according to item 48, wherein the pharmaceutical formulation is diluted before administering it to the patient.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate and does not pose a limitation on the scope unless otherwise claimed. No language in the specification should be construed as indicating any non-claimed element as essential to the practice as used herein.

While exemplary embodiments are provided, it will be understood by those skilled in the art that various changes may be made and equivalents may be substituted for elements thereof without departing from the scope. In addition, many modifications may be made to adapt a particular situation or material to the teachings without departing from the essential scope thereof. Therefore, it is intended that the claims not be limited to the particular embodiment disclosed as the best mode contemplated for carrying out this invention, but that the claims will include all embodiments falling within their scope claims. Any combination of the above-described elements in all possible variations thereof is encompassed unless otherwise indicated herein or otherwise clearly contradicted by context.

1. An aqueous pharmaceutical formulation comprising daptomycin, calcium, and at least one excipient.

2. The aqueous pharmaceutical formulation of claim 1, wherein the at least one excipient does not comprise a buffering agent.

3. The aqueous pharmaceutical formulation according to claim 1, wherein the pH range of the formulation is from 5.5 to 7.5.

4. The aqueous pharmaceutical formulation according to any one of claims 1-4, wherein the calcium is in the form of calcium chloride (CaCl2), Ca-α-D-heptagluconate, calcium saccharin, calcium lactate, or calcium acetate.

5. The aqueous pharmaceutical formulation according to any one of claims 1-5, wherein the calcium is in the form of calcium chloride.

6. The aqueous pharmaceutical formulation according to any one of claims 1-5, wherein the calcium is in the form of calcium saccharin.

7. The aqueous pharmaceutical formulation of any of claims 1 to 6, wherein the calcium is present in a molar ratio to daptomycin of 0.1:1 to 2:1.

8. The aqueous pharmaceutical formulation of claim 6, wherein the calcium is present in a molar ratio to daptomycin of 0.1:1 to 1:1.

9. The aqueous pharmaceutical formulation according to any one of claims 1-8, wherein at least one excipient is selected from organic solvents, such as alkyl alcohols, ethanol, benzyl alcohol, ethylene glycol, propylene glycol, butylene glycol, glycerol, polysorbates, for example polysorbate 20, polysorbate 40, and polysorbate 80, polyalkylene glycols, such as polyethylene glycol (PEG), polyethylene glycol 200 (PEG 200), polyethylene glycol 300 (PEG 300), polyethylene glycol 400 (PEG 400), polyethylene glycol 600 (PEG 600), polypropylene glycol, povidone and polybutylene glycol, and primary amides such as niacinamide.

10. The aqueous pharmaceutical formulation of any one of claims 1-9, wherein the at least one organic solvent comprises at least one of polyethylene glycol (PEG) and/or glycerol.

11. The aqueous pharmaceutical formulation according to any one of claims 9-10, wherein at least one organic solvent is glycerol.

12. The aqueous pharmaceutical formulation according to any one of claims 9-11, wherein at least one organic solvent is polyethylene glycol 400 (PEG 400).

13. The aqueous pharmaceutical formulation according to any one of claims 9-12, wherein the formulation comprises glycerol and PEG 400.

14. The aqueous pharmaceutical formulation according to any one of claims 9-13, wherein the formulation comprises two or more organic solvents.

15. The aqueous pharmaceutical formulation according to any one of claims 9-14, wherein the formulation comprises each organic solvent in the amount of 20% V/V or less.

16. The aqueous pharmaceutical formulation according to any one of claims 9-15, wherein the organic solvent is glycerol which is comprised in the formulation in the amount of 10% V/V or less.

17. The aqueous pharmaceutical formulation according to any one of claims 9-15, wherein the organic solvent is PEG 400 which is comprised in the formulation in the amount of 10% V/V or less.

18. The aqueous pharmaceutical formulation to any of the preceding claims, wherein the daptomycin is at a concentration of from 0.5 mg/mL to 500 mg/mL.

19. The aqueous pharmaceutical formulation of any one of claims from 1-18, wherein the daptomycin is at a concentration of from 2 mg/ml to 20 mg/ml.

20. The aqueous pharmaceutical formulation of any one of claims from 1-19, wherein the daptomycin is at a concentration of 50 mg/ml.

21. An aqueous pharmaceutical formulation comprising 50 mg/ml of daptomycin, wherein the pharmaceutical formulation comprises daptomycin, calcium and PEG 400; wherein the molar ratio of daptomycin to calcium is 1:1, PEG 400 is comprised in concentration of 10% V/V or less and wherein the pH of the formulation is 7.

22. An aqueous pharmaceutical formulation comprising 50 mg/ml of daptomycin, wherein the pharmaceutical formulation comprises daptomycin, calcium, glycerol and PEG 400; wherein the molar ratio of daptomycin to calcium is 1:1, glycerol is comprised in concentration of 10% V/V or less, PEG 400 is comprised in concentration of 10% V/V or less and wherein the pH of the formulation is 7.

23. The aqueous pharmaceutical formulation according to any of claims 1-22, wherein at least one excipient is selected from amino acid, sugar, sugar derivatives, saccharin, organic acids, organic solvents, betaine, taurine, nicotinamide or their pharmaceutically acceptable salts or derivatives thereof.

24. The aqueous pharmaceutical formulation according to any one of claims 1-23, wherein the aqueous pharmaceutical formulation comprises at least 50% water V/V.

25. The aqueous pharmaceutical formulation according to any one of claims 1-24, wherein the aqueous pharmaceutical formulation comprises more than 50% water V/V.

26. The aqueous pharmaceutical formulation according to any one of claims 1-25, wherein the aqueous pharmaceutical formulation comprises at least 60% water V/V.

27. The aqueous pharmaceutical formulation according to any one of claims 1-24, wherein the aqueous pharmaceutical formulation comprises at least 50%, 60%, 70%, 80%, 85%, 90%, 95%, 98%, or 99% water V/V.

28. The aqueous pharmaceutical formulation of any one of claims from 1-27, wherein the formulation is packaged in a vial for dilution prior to administration a patient.

29. The aqueous pharmaceutical formulation according to any one of claims from 1-28, wherein the aqueous pharmaceutical formulation is stable for at least 4 days at temperatures of 30° C.

30. The aqueous pharmaceutical formulation according to any of the preceding claims, for use in treatment of microbial infections caused by Gram positive bacteria.

31. The aqueous pharmaceutical formulation according to claim 30, for use in treatment of skin and soft-tissue infections (cSSTI) or Staphylococcus aureus bloodstream infections (bacteremia).

32. The process for manufacturing aqueous pharmaceutical formulations according to any of the preceding claims, comprising steps of mixing of daptomycin, calcium and at least one excipient into solution, adjusting the pH of such solution to pH from 5.5 to 7.5 with a suitable pH adjusting agent.

33. A method of treating a patient with a microbial infection comprising administering the aqueous pharmaceutical formulation according to any one of claims 1-32, optionally diluting the pharmaceutical formulation before administering it to the patient.

34. The method of treating a patient according to claim 33, wherein the pharmaceutical formulation is diluted before administering it to the patient.
